SEQUENCE LISTING (21 sequences)

SEQ ID NO: 1 - DNA, 1611 bp, Homo sapiens
  CDS (1)..(1611)
atgggtagcaacaagagcaagcccaaggatgccagccagcggcgccgcagcctggagcccgccgagaacgtgcacggcgctggcgggggcgctttccccgcctcgcagacccccagcaagccagcctcggccgacggccaccgcggccccagcgcggccttcgcccccgcggccgccgagcccaagctgttcggaggcttcaactcctcggacaccgtcacctccccgcagagggcgggcccgctggccggtggagtgaccacctttgtggccctctatgactatgagtctaggacggagacagacctgtccttcaagaaaggcgagcggctccagattgtcaacaacacagagggagactggtggctggcccactcgctcagcacaggacagacaggctacatccccagcaactacgtggcgccctccgactccatccaggctgaggagtggtattttggcaagatcaccagacgggagtcagagcggttactgctcaatgcagagaacccgagagggaccttcctcgtgcgagaaagtgagaccacgaaaggtgcctactgcctctcagtgtctgacttcgacaacgccaagggcctcaacgtgaagcactacaagatccgcaagctggacagcggcggcttctacatcacctcccgcacccagttcaacagcctgcagcagctggtggcctactactccaaacacgccgatggcctgtgccaccgcctcaccaccgtgtgccccacgtccaagccgcagactcagggcctggccaaggatgcctgggagatccctcgggagtcgctgcggctggaggtcaagctgggccagggctgctttggcgaggtgtggatggggacctggaacggtaccaccagggtggccatcaaaaccctgaagcctggcacgatgtctccagaggccttcctgcaggaggcccaggtcatgaagaagctgaggcatgagaagctggtgcagttgtatgctgtggtttcagaggagcccatttacatcgtcacggagtacatgagcaaggggagtttgctggactttctcaagggggagacaggcaagtacctgcggctgcctcagctggtggacatggctgctcagatcgcctcaggcatggcgtacgtggagcggatgaactacgtccaccgggaccttcgtgcagccaacatcctggtgggagagaacctggtgtgcaaagtggccgactttgggctggctcggctcattgaagacaatgagtacacggcgcggcaaggtgccaaattccccatcaagtggacggctccagaagctgccctctatggccgcttcaccatcaagtcggacgtgtggtccttcgggatcctgctgactgagctcaccacaaagggacgggtgccctaccctgggatggtgaaccgcgaggtgctggaccaggtggagcggggctaccggatgccctgcccgccggagtgtcccgagtccctgcacgacctcatgtgccagtgctggcggaaggagcctgaggagcggcccaccttcgagtacctgcaggccttcctggaggactacttcacgtccaccgagccccagtaccagcccggggagaacctctag

SEQ ID NO: 2 - protein, 536 aa, Homo sapiens
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 3 - DNA, 27 bp, Artificial Sequence
  Sense Oligonucleotide
atgtccccagaggccttcctgcaggac

SEQ ID NO: 4 - DNA, 44 bp, Artificial Sequence
  Oligonucleotide
ttaaatcctaggttctccccgggctcgtactgtggctcagtgga

SEQ ID NO: 5 - protein, 9 aa, Homo sapiens
  MISC_FEATURE (5)..(5): “ser”  is phosphorylated
QRRRSLEPA

SEQ ID NO: 6 - protein, 11 aa, Homo sapiens
  MISC_FEATURE (6)..(6): “ser” is phosphorylated
SQRRRSLEPAE

SEQ ID NO: 7 - protein, 13 aa, Homo sapiens
  MISC_FEATURE (7)..(7): “ser” is phosphorylated
ASQRRRSLEPAEN

SEQ ID NO: 8 - protein, 15 aa, Homo sapiens
  MISC_FEATURE (8)..(8): “ser” is phosphorylated
DASQRRRSLEPAENV

SEQ ID NO: 9 - protein, 17 aa, Homo sapiens
  MISC_FEATURE (9)..(9): “ser” is phosphorylated
KDASQRRRSLEPAENVH

SEQ ID NO: 10 - protein, 19 aa, Homo sapiens
  MISC_FEATURE (10)..(10): “ser” is phosphorylated
PKDASQRRRSLEPAENVHG

SEQ ID NO: 11 - protein, 21 aa, Homo sapiens
  MISC_FEATURE (11)..(11): “ser” is phosphorylated
KPKDASQRRRSLEPAENVHGA

SEQ ID NO: 12 - protein, 17 aa, Homo sapiens
KDASQRRRSLEPAENVH

SEQ ID NO: 13 - protein, 17 aa, Artificial Sequence
  Protein Sequence
KDAAQRRRSLEPAENVH

SEQ ID NO: 14 - protein, 17 aa, Artificial Sequence
  Protein Sequence
KDASQRRRALEPAENVH

SEQ ID NO: 15 - protein, 17 aa, Artificial Sequence
  Protein Sequence
KDAAQRRRALEPAENVH

SEQ ID NO: 16 - protein, 17 aa, Artificial Sequence
  Protein Sequence
KDASQRRRDLEPAENVH

SEQ ID NO: 17 - protein, 17 aa, Artificial Sequence
  Protein Sequence
KDASQARRSLEPAENVH

SEQ ID NO: 18 - protein, 17 aa, Artificial Sequence
  Protein Sequence
KDASQRARSLEPAENVH

SEQ ID NO: 19 - protein, 17 aa, Artificial Sequence
  Protein Sequence
KDASQRRASLEPAENVH

SEQ ID NO: 20 - protein, 17 aa, Artificial Sequence
  Protein Sequence
HVNESPELSAAAQSADK

SEQ ID NO: 21 - protein, 16 aa, Artificial Sequence
  Flag; Protein Sequence
KDDDDKYDKDDDDKYD

(19) United States
(12) Patent Application Publication
(10) Pub. No.: US 2005/0037446 A1
(43) Pub. Date: Feb. 17, 2005

(54) Agents that recognize src when phosphorylated at serine 17

(76) Inventors: John Michael II Schmitt, Beaverton, OR (US); Philip J.S. Stork, Portland, OR (US)

Correspondence Address: KLARQUIST SPARKMAN, LLP, 121 SW SALMON STREET, SUITE 1600, PORTLAND, OR, 97204, US

(21) Appl. No.: 10/497,641

(22) PCT Filed: Dec. 27, 2002
(86) PCT No.: PCT/US02/41564

Related U.S. Application Data
(60) Provisional application No. 60/345,888, filed on Dec. 28, 2001.

(51) Int. Cl. G01N 33/574; A61K 39/395; C07K 16/30

(52) U.S. Cl. 435/7.23; 530/388.8; 530/388.26

(57) ABSTRACT

Specific binding agents that specifically recognize Src when it is phosphorylated at Ser17, and methods of their use, are disclosed. Methods of identifying abnormal cell proliferation by detecting a mutation in Src at Ser17 are disclosed. Methods of screening agents which alter proliferation, or which alter phosphorylation of Src at Ser17, are disclosed. Methods of altering cellular proliferation by altering phosphorylation of Src at Ser17 are disclosed.

### CROSS REFERENCE TO RELATED APPLICATION

This application claims priority to U.S. Provisional Application No. 60/345,888 filed Dec. 28, 2001, hereby incorporated by reference in its entirety.

### ACKNOWLEDGEMENT OF GOVERNMENT SUPPORT

This invention was made with Government support under grants NCI CA072971-04 and NIH T32 HL07781. Therefore, the Government may have certain rights to the invention.

### FIELD

This application relates to specific binding agents that recognize Src when it is phosphorylated at serine-17 (Ser17), and methods of using such agents for diagnosis and treatment of various disorders.

### BACKGROUND

The viral Src gene (v-Src), which is responsible for the development of tumors in chickens, was the first retroviral oncogene to be identified. The cellular homolog, c-Src, has also been implicated in the development, growth, progression and metastasis of several cancers, including those of the colon, breast, pancreas, and brain (Irby and Yeatman, Oncogene 19:5636-42, 2000). Src protein kinase activity is frequently elevated in neoplastic tissues when compared to adjacent normal tissues. Although it is hypothesized that Src may play a significant role in these processes that affect cancer outcome, the actual mechanism has not been identified.

Over twenty years ago it was suggested that Src was a direct substrate of PKA. An increase in phospho-serine within Src's amino-terminus was observed following treatment with cAMP (Collett et al., Cell 15:1363-9, 1978; Collett et al., J. Virol. 29:770-81, 1979), which increased the kinase activity of Src (Roth et al., J. Biol. Chem. 272:30806-11, 1983). A consensus PKA site at Ser17 of both v-Src and c-Src was proposed to be the major site of serine phosphorylation in both Src proteins (Takeya et al., J. Virol. 44:1-11, 1982). Although deletion of amino acids 15-27 within v-Src reduced serine phosphorylation of v-Src, this did not interfere with constitutive kinase activity or oncogenicity of the mutant v-Src protein (Cross and Hanafusa, Cell 34:597-607, 1983), and no physiological role for phosphorylation at Ser17 has since been proposed (Brown and Cooper, Biochim. Biophys. Acta 1287:121-49, 1996).

Intracellular signaling by second messengers has played a central role in understanding of cell proliferation for many decades (Ryan and Heidrick, Science 162:1484-5, 1968). The anti-proliferative action of cAMP has been studied in conjunction with hormone receptors linked to Gs, adenylyl cyclase, and the activation of the cyclic AMP-dependent protein kinase PKA (Beavo et al., Proc. Natl. Acad. Sci. USA 71:3580-3, 1974). Because of these growth inhibitory effects, strategies to regulate cAMP and PKA have been proposed as anti-tumor therapies (Cho-Chung et al., Front. Biosci. 4: D898-907, 1999; Pastan et al., Anne Rev. Biochem. 44:491-522, 1975; Puck, Proc. Natl. Acad. Sci. USA 74:4491-5, 1977; Tortora et al., Clin. Cancer Res. 1:377-84, 1995). One candidate mediating cAMP's inhibition of Raf-1 is the small G protein, Rap1 (Dugan et al., J. Biol. Chem. 274:25842-8, 1999; Schmitt and Stork, Mol. Cell. Biol. 21: 3671-83, 2001; Tsygankova et al., Mol. Cell. Biol. 21: 1921-9,2001). Rap1 is a ubiquitously expressed small GTP-binding protein that is activated by cAMP. Despite abundant literature on cAMP's ability to activate Rap1 (Altschuler et al., J. Biol. Chem. 270: 10373-6, 1995; Chen et al., Cancer Res. 59:213-8, 1999; de Rooij et al., Nature 396:474-7, 1998; Dugan et al., J. Biol. Chem. 274: 25842-8, 1999; Seidel et al., J. Biol. Chem. 274:2583341, 1999; Tsygankova et al., Mol. Cell. Biol. 21:1921-9, 2001; von Lintig et al., Oncogene 19:4029-34, 2000; Vossler et al., Cell 89:73-82, 1997; Wan and Huang, J. Biol. Chem. 273:14533-7, 1998; Zanassi et al., J. Biol. Chem 276:11487-95, 2001), the mechanism of cAMP's activation of Rap1 is unknown.

Therefore, there is a need to identify the mechanism by which Src mediates its proliferative effects, and the mechanism by which cAMP activates Rap1, so that therapies to treat proliferative disorders, or disorders in which proliferation is desired, can be developed. In addition, identification of the mechanism will permit development of alternative diagnostics, which may allow the design of better treatment regimens.

### SUMMARY

This disclosure for the first time demonstrates a role for Src in mediating PKA's antiproliferative effects. Specifically, the role of phosphorylating Src at serine at position 17 (Ser17) on cellular proliferation is disclosed. Phosphorylation of Src Ser17 has anti-proliferative effects, while mutants that cannot be phosphorylated at Src Ser17, such as the mutant Src S17A, have proliferative effects.

Agents are disclosed that specifically bind to Src when Src is phosphorylated at Ser17, but do not bind when Ser17 is not phosphorylated Examples of such agents include antibodies and other proteins, as well as non-protein drugs. In addition, methods are disclosed for detecting Src when it is or is not phosphorylated at Ser17.

Also disclosed is a method of identifying a cell having abnormal proliferation, such as increased or decreased proliferation, by detecting a mutation in Src at Ser17 that is associated with abnormal cellular proliferation.

Methods are also disclosed for screening one or more test agents to identify agents that alter cell proliferation. For example, the test agent is contacted with a cell in which Src is mutated at Ser17, and a determination is made to determine whether the test agent alters the abnormal cell proliferation. Examples of assays that can be used to make such a determination include, but are not limited to, a proliferation assay, a Rap1 activation assay, or a Ras activation assay. Also disclosed is a method of screening one or more test agents to identify agents that alter phosphorylation of Src at Ser17. The method includes contacting the test agents with a Src peptide that includes Ser17, performing a kinase assay, and determining whether the test agent enhanced Src phosphorylation at Ser17.

A method of altering cell proliferation is disclosed. The method includes altering phosphorylation of Src Ser17 in a cell, for example by contacting the cell with an agent that alters phosphorylation of Src Ser17.

Transgenic mammals, such as humans and mice, that have a mutation at Src Ser17 that alters phosphorylation of Src Ser17, are disclosed, as well as methods of using the transgenic mammals to screen for agents that affect cellular proliferation.

The foregoing and other objects, features, and advantages of the agents and methods described herein will become more apparent from the following detailed description of several embodiments, which proceeds with reference to the accompanying figures.

### BRIEF DESCRIPTION OF THE FIGURE

FIG. 1 is a schematic diagram of cAMP's inhibition of ERKs and cell growth

### SEQUENCE LISTING

The nucleic and amino acid sequences in the accompanying sequence listing are shown using standard letter abbreviations for nucleotides, and three letter code for amino acids. Only one strand of each nucleic acid sequence is shown, but the complementary strand is understood as included by any reference to the displayed strand.

SEQ ID NO: 1 is a nucleic acid sequence showing a cDNA sequence of human c-Src. (Genbank Accession Number NM_{—}005417).

SEQ ID NO: 2 is an amino acid sequence showing a human c-Src sequence (Genbank Accession Number P12931).

SEQ ID NOS: 3 and 4 are nucleic acid sequences of primers that can be used to PCR amplify Src.

SEQ ID NOS: 5-11 are examples of peptide sequences that can be used to generate phosphospecific antibodies that recognize Src Ser17 when phosphorylated.

SEQ ID NO: 12 is a peptide sequence of wild-type Src.

SEQ ID NO: 13 is the peptide sequence of the Src S12A mutant.

SEQ ID NO: 14 is the peptide sequence of the Src S17A mutant.

SEQ ID NO: 15 is the peptide sequence of the Src S12AS17A mutant.

SEQ ID NO: 16 is the peptide sequence of the Src S17D mutant.

SEQ ID NO: 17 is the peptide sequence of the Src R14A mutant.

SEQ ID NO: 18 is the peptide sequence of the Src R15A mutant.

SEQ ID NO: 19 is the peptide sequence of the Src R16A mutant.

SEQ ID NO: 20 is the peptide sequence of a scrambled peptide.

SEQ ID NO: 21 is the peptide sequence of a Flag peptide.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein, “comprising” means” “including” and the singular forms “a” or “an” or “the” include plural references unless the context clearly dictates otherwise. For example, reference to “comprising a protein” includes one or a plurality of such proteins, and reference to “comprising the antibody” includes reference to one or more antibodies and equivalents thereof, and so forth.

Unless explained otherwise, all technical and scientific terms used herein have the same meaning as commonly understood to one of ordinary skill in the art to which this disclosure belongs.

Agent: Any substance, including, but not limited to, an antibody, chemical compound, molecule, peptidomimetic, or protein.

Amplify: To increase in number; for example DNA molecules can be amplified by in vitro amplification. In vitro amplification includes techniques that increase the number of copies of a nucleic acid molecule in a sample or specimen. An example of amplification is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid. The product of in vitro amplification can be characterized by electrophoresis, restriction endonuclease cleavage patterns, oligonucleotide hybridization or ligation, and/or nucleic acid sequencing, using standard techniques. Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Animal: Living multicellular vertebrate organisms, a category which includes, for example, non-human and human mammals.

Antibody: An immunoglobulin molecule including an antigen-binding site that specifically binds (immunoreacts with) an antigen. Examples include polyclonal antibodies, monoclonal antibodies, humanized monoclonal antibodies, chimeric antibodies, and immunologically active portions thereof.

Naturally occurring antibodies (e.g., IgG) include four polypeptide chains, two heavy (H) chains and two light (L) chains inter-connected by disulfide bonds. However, the antigen-binding function of an antibody can be performed by fragments of a naturally occurring antibody. Immunologically effective portions of monoclonal antibodies include, but are not limited to: Fab, Fab′, F(abt)₂, Fabc and Fv portions (for a review, see Better and Horowitz, Methods. Enzymol. 1989, 178:476-96). Other examples of antigen-binding fragments include, but are not limited to: (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment which consists of a VH domain; (v) an isolated complimentarity determining region (CDR); and (vi) an F(ab′)₂ fragment, a bivalent fragment comprising two Fab fragments linked by a disulfide bridge at the hinge region. Furthermore, although the two domains of the Fv fragment are coded for by separate genes, a synthetic linker can be made that enables them to be made as a single protein chain (known as single chain Fv (scFv) by recombinant methods. Such single chain antibodies are also included.

Antigen: A compound, composition, or substance that can stimulate the production of antibodies or a T-cell response in an animal, including compositions that are administered, such as injected or absorbed, to an animal. An antigen reacts with the products of specific humoral or cellular immunity, including those induced by heterologous immunogens. The term “antigen” includes all related antigenic epitopes.

Antineoplastic agent: A drug or biologic that decreases the proliferation of neoplastic cells, for example arresting their growth or causing regression of a tumor. Examples include alkylating agents (such a vincristine, vinblastine or taxol), anthracycline antibiotics such as daunorubicin and doxorubicin, hormonal therapies such as tamoxifen, and miscellaneous agents such as cis-diamminedichloroplatimun (II), and hydroxyurea. Antineoplastic agents also include biologics, such as IL-2 and alpha-interferon, and immunotherapy, for example with bacille Calmette-Guerin (BCG). Protocols for administration of such agents are known in the art, and examples can be found in Goodman and Gilman, The Pharmacological Basis of Therapeutics, 17^{th} edition, section XIII.

Cancer: Malignant neoplasm that has undergone characteristic anaplasia with loss of differentiation, increased rate of growth, invasion of surrounding tissue, and is capable of metastasis.

Chemical synthesis: An artificial means by which one can make a protein or peptide, for example as described in EXAMPLE 22.

cDNA (complementary DNA): A piece of DNA lacking internal, non-coding segments (introns) and regulatory sequences which determine transcription. cDNA can be synthesized in the laboratory by reverse transcription from messenger RNA extracted from cells.

Conservative substitution: One or more amino acid substitutions for amino acid residues having similar biochemical properties. Typically, conservative substitutions have little to no impact on the activity of a resulting polypeptide. For example, a conservative substitution is an amino acid substitution in a Src peptide that does not substantially affect the ability of the Src peptide to modulate proliferation, such as increase or decrease proliferation, depending on the phosphorylation state of Ser17. In a particular example, a conservative substitution is an amino acid substitution in a Src peptide, such as a conservative substitution in SEQ ID NO: 2, 13 or 16, that does not significantly alter the ability of the protein to modulate proliferation (for example, a conservative substitution in SEQ ID NO: 16 (Src S17D) will not significantly decrease the anti-proliferative activity of Src S17D). Methods that can be used to determine the amount of proliferation by a variant Src peptide are disclosed herein (for example, the Mu assay described in EXAMPLE 3). An alanine scan can be used to identify which amino acid residues in a Src peptide can tolerate an amino acid substitution. In one example, proliferation is not altered by more than 25%, for example not more than 20%, for example not more than 10%, when an alanine, or other conservative amino acid (such as those listed below), is substituted for one or more native amino acids.

In one example, one conservative substitution is included in the peptide, such as a conservative substitution in SEQ ID NO: 2, 13 or 16. In another example, two or less conservative substitutions are included in the peptide. In a further example, three or less conservative substitutions are included in the peptide. A polypeptide can be produced to contain one or more conservative substitutions by manipulating the nucleotide sequence that encodes that polypeptide using, for example, standard procedures such as site-directed mutagenesis or PCR Alternatively, a polypeptide can be produced to contain one or more conservative substitutions by using standard peptide synthesis methods.

Substitutional variants are those in which at least one residue in the amino acid sequence has been removed and a different residue inserted in its place. Examples of amino acids which may be substituted for an original amino acid in a protein and which are regarded as conservative substitutions include: Ser for Aaa; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

Further information about conservative substitutions can be found, among other references, in Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988) and in standard textbooks of genetics and molecular biology.

Deletion: The removal of a nucleic acid or amino acid sequence, for example DNA, the regions on either side being joined together.

Detectable: Capable of having an existence or presence ascertained. For example, binding of an antibody to an antigen is detectable if the signal generated from such binding is strong enough to be measurable.

DNA: Deoxyribonucleic acid. DNA is a long chain polymer which comprises the genetic material of most living organisms (some viruses have genes comprising ribonucleic acid, RNA). The repeating units in DNA polymers are four different nucleotides, each of which comprises one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Degenerate variant: A polynucleotide encoding a Src polypeptide that includes a sequence that is degenerate as a result of the genetic code. There are 20 natural amino acids, most of which are specified by more than one codon. Therefore, all degenerate nucleotide sequences are included as long as the amino acid sequence of the Src polypeptide encoded by the nucleotide sequence is unchanged.

Drug or Pharmaceutical agent: Any natural or artificially made chemical compound or composition, that can be used on or administered to a subject (e.g. humans or animals) as an aid in the diagnosis, treatment or prevention of a disease or other abnormal condition.

Enhance: To improve the quality, amount, or strength of something. In one example, a therapy enhances cell proliferation if cell proliferation increases in the presence of the therapy. In a particular example, a Src mutant that has a reduced ability to be phosphorylated at Ser 17, enhances cell proliferation. Such enhancement can be measured, for example, using an MTT assay.

Functionally Equivalent: Having an equivalent function. In the context of a Src molecule, functionally equivalent molecules include different molecules that retain the function of Src. For example, functional equivalents can be provided by sequence alterations in a Src or Src mutant, wherein the peptide with one or more sequence alterations retains a function of the unaltered peptide, such that it retains its ability to modulate proliferation, such as the ability to increase proliferation (for Src S17A) or the ability to decrease proliferation (for phosphorylated Ser-17 Src, or Src S17D).

Examples of sequence alterations include, but are not limited to, conservative substitutions, deletions, mutations, frameshifts, and insertions. In one example, a given polypeptide binds an antibody, and a functional equivalent is a polypeptide that binds the same antibody. Thus a functional equivalent includes peptides that have the same binding specificity as a polypeptide, and that can be used as a reagent in place of the polypeptide (such as in a diagnostic assay). In one example a functional equivalent includes a polypeptide wherein the binding sequence is discontinuous, wherein the antibody binds a linear epitope. Thus, if the peptide sequence is QRRRSLEPA (SEQ ID NO: 5) a functional equivalent includes discontinuous epitopes, that can appear as follows (**=any number of intervening amino acids): NH2-**-Q**R**R**R**S**L**E**P**A-COOH.

In this example, the polypeptide is functionally equivalent to SEQ ID NO: 5 if the three dimensional structure of the polypeptide is such that it can bind a monoclonal antibody that binds SEQ ID NO: 5.

Hybridoma: A single-cell cloned cell that secretes a homogenous population of monoclonal antibodies.

Isolated: An “isolated” biological component (such as a nucleic acid or protein) has been substantially separated, produced apart from, or purified away from other biological components in the cell of the organism in which the component naturally occurs, i.e., other chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids, proteins and peptides.

Label: An agent that generates an amount of detectable signal. Examples of labels include, but are not limited to fluorophores, chemiluminescent molecules, radioactive isotopes, ligands, and enzymes. Methods for labeling and guidance in the choice of labels appropriate for various purposes are discussed, e.g., in Sambrook et al., Molecular Cloning: A Laboratory Manual, Cold Spring Harbor Laboratory Press (1989) and Ausubel et al., Current Protocols in Molecular Biology, Greene Publishing Associates and Wiley-Intersciences (1987).

Malignant: Cells that have the properties of anaplasia invasion and metastasis.

Neoplasm: Abnormal growth of cells.

Normal Cell: A cell having no pathological characteristics. Examples include non-tumor cells, non-malignant cells, non-diseased, or uninfected cells. In one example, normal cells are cells obtained from a normal subject, for example as compared to a subject having a cancer.

Normal Subject: A subject who does not have a proliferative disorder, such as cancer, osteoporosis, or heart disease.

Oligonucleotide: A linear polynucleotide (such as DNA or RNA) sequence of at least 9 nucleotides, for example at least 15, 18, 24, 25, 27, 30, 50, 100 or even 200 nucleotides long.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF (open reading frame): A series of nucleotide triplets (codons) coding for amino acids without any termination codons. These sequences are usually translatable into a peptide.

Ortholog: Two nucleotide sequences are orthologs of each other if they share a common ancestral sequence and diverged when a species carrying that ancestral sequence split into two species. Orthologous sequences are also homologous sequences.

PCR (polymerase chain reaction): A technique in which cycles of denaturation, annealing with primer, and then extension with DNA polymerase are used to amplify the number of copies of a target DNA sequence.

Peptide Modifications: The present disclosure includes Src peptides and variants thereof, as well as synthetic embodiments of the peptides described herein. In addition, analogues (non-peptide organic molecules), derivatives (chemically functionalized peptide molecules obtained starting with the disclosed peptide sequences) and variants (homologs) of these proteins that modulate proliferation, such as increase or decrease cell proliferation, can be utilized in the methods described herein. The peptides disclosed herein include a sequence of amino acids, tha can be either L- and/or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₋₆ alkyl, or combined to form a heterocyclic ring, such as a 5- or 6 membered ring. Amino groups of the peptide, whether amino-terminal or side chain, may be in the form of a pharmaceutically-acceptable acid addition salt, such as the HCl, HBr, acetic, benzoic, toluene sulfonic, maleic, tataric and other organic salts, or may be modified to C₁-C₁₆ alkyl or dialkyl amino or further converted to an amide. Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as F, Cl, Br or I, or with Cl-C₁₋₆ alkyl, Cl-C₁₋₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability. For example, a C- or N-terminal cysteine can be added to the peptide, so that when oxidized the peptide will contain a disulfide bond, generating a cyclic peptide. Other peptide cyclizing methods include the formation of thioethers and carboxyl- and amino-terminal amides and esters.

Peptidomimetic and organomimetic embodiments are also within the scope of the present disclosure, whereby the three-dimensional arrangement of the chemical constituents of such peptido- and organomimetics mimic the three-dimensional arrangement of the peptide backbone and component amino acid side chains, resulting in such peptido- and organomimetics of the proteins of this invention having measurable or enhanced ability to modulate proliferation. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharmacology Munson (ed.) 1995, Ch. 102, for descriptions of techniques used in CADD. Also included within the scope of the disclosure are mimetics prepared using such techniques. In one example, a mimetic mimics the modulation in cell proliferation generated by Src or a mutant thereof.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975) and U.S. Pat. No. 6,034,114, describes compositions and formulations suitable for pharmaceutical delivery of the peptides herein disclosed.

In general, the nature of the carrier will depend on the particular mode of administration being employed. For instance, parenteral formulations usually comprise injectable fluids-that include pharmaceutically and physiologically acceptable fluids such as water, physiological saline, balanced salt solutions, aqueous dextrose, glycerol or the like as a vehicle. For solid compositions (e.g. powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phosphospecific antibody: An antibody that specifically binds to a particular protein or peptide when phosphorylated, at a particular amino acid. In one example, it is an antibody that specifically recognizes and binds to Src when it is phosphorylated at Ser17, and does not recognize other proteins that may or may not be phosphorylated. In particular examples, such antibodies can be used for diagnosis, for example to diagnose and/or follow the prognosis of a Src-positive tumor.

Polynucleotide: A linear nucleic acid sequence of any length. Therefore, a polynucleotide includes molecules that are at least 15, 24, 27, 30, 50, 100, 200, 500, 1000, or 5000 nucleotides in length, and also nucleotides as long as a full length cDNA. A Src polynucleotide encodes a Src peptide.

Polypeptide: Any chain of amino acids at least six amino acids in length, such as at least 8 amino acids, such as at least 9 amino acids, regardless of post-translational modification (e.g., glycosylation or phosphorylation). In one example, a polypeptide is a Src peptide or mutant thereof, such as SEQ ID NO: 2, 13 or 16.

Preventing or treating a disease: “Preventing” a disease refers to inhibiting the full development of a disease, for example preventing development or metastasis of a tumor in a person having a Src-positive tumor, or for example preventing progression of a disease in which increase cell proliferation is desired such as heart disease or osteoporosis. “Treatment” refers to a therapeutic intervention that ameliorates a sign or symptom of a disease or pathological condition, such as conditions related to the presence of a Src-expressing tumor, for example halting the progression of a tumor, reducing the size of the tumor, or even elimination of the tumor.

Proliferation: To increase in number or amount, for example increasing a number of cells. In one example, proliferation is enhanced or increased in the presence of an agent, when compared to the absence of the agent, if in the presence of the agent an increase in proliferation is observed. In another example, proliferation is reduced or decreased in the presence of an agent, when compared to the absence of the agent, if in the presence of the agent a decrease or reduction in proliferation is observed. Cell proliferation can be measured using any assay known in the art, for example the MTT assay described in EXAMPLE 3. Promoter: An array of nucleic acid control sequences that direct transcription of a nucleic acid. A promoter includes necessary nucleic acid sequences near the start site of transcription, such as, in the case of a polymerase 1: type promoter, a TATA element A promoter also optionally includes distal enhancer or repressor elements that can be located as much as several thousand base pairs from the start site of transcription.

Purified: The term purified does not require absolute purity; rather, it is intended as a relative term. Thus, for example, a purified peptide preparation is one in which the peptide or protein is more enriched than the peptide or protein is in its environment within a cell, such that the peptide is substantially separated from cellular components (nucleic acids, lipids, carbohydrates, and other polypeptides) that may accompany it. In another example, a purified peptide preparation is one in which the peptide is substantially-free from contaminants, such as those that might be present following chemical synthesis of the peptide.

In one example, a Src peptide is purified when at least 60% by weight of a sample is composed of the peptide, for example when at least 75%, 95%, or 99% or more of a sample is composed of the peptide. Examples of methods that can be used to purify an antigen, include, but are not limited to the methods disclosed in Sambrook et al. (Molecular Cloning: A Laboratory Manual, Cold Spring Harbor, N.Y., 1989, Ch. 17). Protein purity can be determined by, for example, polyacrylamide gel electrophoresis of a protein sample, followed by visualization of a single polypeptide band upon staining the polyacrylamide gel; high-pressure liquid chromatography; sequencing; or other conventional methods.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Sample: A material to be analyzed. In one example, a sample is a biological sample. In one embodiment, a biological sample contains genomic DNA, cDNA, RNA, or protein obtained from the cells of a subject Other examples of biological samples, include, but are not limited to: peripheral blood, serum, plasma, urine, cerebrospinal fluid, pleural fluid, synovial fluid, peritoneal fluid, gastric fluid, saliva, lymph fluid, interstitial fluid, sputum, stool physiological secretions, tears mucus, sweat, milk, semen, seminal fluid, vaginal secretions, fluid from ulcers and other surface eruptions, blisters, and abscesses, tissue biopsy, surgical specimen, fine needle aspriates, amniocentesis samples and autopsy material.

Sequence identity: The similarity between amino acid sequences, or two nucleic acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a Src peptide, disclosed herein, will possess a relatively high degree of sequence identity when aligned using standard methods.

Methods of alignment of sequences for comparison are well known in the art. Various programs and alignment algorithms are described in: Smith & Waterman Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al, J. Mol. Biol. 215:403-10, 1990) is available from several sources, including the National Center for Biological Information (NCBI, National Library of Medicine, Building 38A, Room 8N₈O₅, Bethesda, Md. 20894) and on the Internet, for use in connection with the sequence analysis programs blastp, blastn, blastx, tblastn and tblastx.

For comparisons of amino acid sequences of greater than about 30 amino acids, the Blast 2 sequences function is employed using the default BLOSUM62 matrix set to default parameters, (gap existence cost of 11, and a per residue gap cost of 1). Src homologs are typically characterized by possession of at least 70% sequence identity counted over the full-length alignment with a Src amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity to Src sequences will show increasing percentage identities when assessed by this method, such as at least 75%, 80%, 85%, 90%, 95%, or 99% sequence identity.

Variants of a Src peptide are typically characterized by possession of at least 50% sequence identity counted over the full length alignment with the amino acid sequence of Src using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 90%, at least 95%, at least 98%, or even at least 99% sequence identity. When less than the entire sequence is being compared for sequence identity, homologs and variants will typically possess at least 80% sequence identity over short windows of 10-20 amino acids, and may possess sequence identities of at least 85%, at least 90%, at least 95%, or 98% depending on their similarity to the reference sequence. Methods for determining sequence identity over such short windows are described at the website that is maintained by the National Center for Biotechnology Information in Bethesda, Md.

One indication that two nucleic acid molecules are closely related is that the two molecules hybridize to each other under stringent conditions. Stringent conditions are sequence-dependent and are different under different environmental parameters. Nucleic acid molecules that hybridize under stringent conditions to a Src gene sequence typically hybridize to a probe based on either an entire Src gene or selected portions of the gene, respectively, under conditions described in EXAMPLE 19. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least 60%, 70%, 80%, 90%, 95%, 98%, or 99% sequence identity determined by this method.

One of skill in the art will appreciate that these sequence identity ranges are provided for guidance only; it is entirely possible that strongly significant homologs could be obtained that fall outside of the ranges provided.

Specific binding agent: An agent that binds substantially only to a defined target. Examples of specific binding agents include antibodies (such as monoclonal or polyclonal antibodies) receptors (such as soluble receptors) and non-peptide chemical compounds. For example, a phosphospecific Src Ser17 binding agent includes anti-Src antibodies and other agents (such as a peptide or drug) that bind substantially to only a Src protein when it is phosphorylated at Ser17.

Specifically binds: The ability of a specific binding agent to specifically react with a particular analyte, for example to specifically immunoreact with an antibody, or to specifically bind to a particular peptide sequence. The binding is a non-random binding reaction, for example between an antibody molecule and an antigenic determinant Binding specificity of an antibody is typically determined from the reference point of the ability of the antibody to differentially bind the specific antigen and an unrelated antigen, and therefore distinguish between two different antigens, particularly where the two antigens have unique epitopes. An antibody that specifically binds to a particular epitope is referred to as a “specific antibody.”

Src: Includes any c-Src gene, cDNA, RNA, or protein from any organism. In one example, Src includes mammalian Src sequences. In another example, a Src sequence includes a fill-length wild-type sequence, as well as shorter sequences that retain the ability to inhibit cell proliferation when Ser17 is phosphorylated. In yet another example, a Src sequence includes a full-length wild-type sequence, as well as shorter sequences that retain the ability to increase cell proliferation when Ser17 is not phosphorylated. This description includes natural Src allelic variants, as well as any variant, fragment, or fusion sequence that retains the ability to modulate cell proliferation, in any species. Non-limiting specific examples include mouse, rat, chicken, rabbit, cat, and human Src.

Src biological activity: The ability of Src to modulate cell proliferation, such as increase or decrease cell proliferation. In one example, it is the ability of Src to decrease cell proliferation when Ser17 is phosphorylated. In another example, it is the ability of Src to increase cell proliferation when Ser 7 is not phosphorylated. Cell proliferation can be measured using any assay known in the art, for example the MTT assay described in EXAMPLE 3.

In other examples, Src biological activity is the ability of Src to activate Rap1 when Ser17 is phosphorylated. In yet other examples, it is the ability of Src to inhibit growth factor stimulation of ERKs, when Ser17 is phosphorylated. In still other examples, it is the ability of Src to assemble a Cb1/Crk/C3G complex, when Ser17 is phosphorylated.

Subject: Includes any mammalian subject, such as a human or veterinary subject.

Test agent: A molecule or composition whose effect on cell proliferation and/or phosphorylation of Src Ser17 is desired to assay. The test agent can be any molecule or mixture of molecules, optionally in a suitable carrier. Examples include, but are not limited to pharmaceutical agents or drugs, such as biological materials (e.g. proteins, nucleic acids, and antibodies).

Therapeutically Effective Amount: Therapeutic preparations disclosed herein are administered in therapeutically effective amounts. An effective amount is that amount of a pharmaceutical preparation that alone, or together with further doses or other agents, stimulates the desired response.

In one example, a desired response is modulation of cell proliferation, such as increasing or decreasing (such as inhibiting) cell proliferation. In particular examples, the desired response is decreasing cell proliferation of a Src-expressing or over-expressing tumor, resulting in halting or slowing the progression of, or inducing a regression of, a pathological condition or which is capable of relieving signs or symptoms caused by the condition. One example of a therapeutic effect is regression of a tumor. Treatment can involve only slowing the progression of the disease temporarily, but can also include halting or reversing the progression of the disease permanently. In another example, a therapeutically effective amount is an amount sufficient to increase the efficacy of another agent, such as an anti-neoplastic agent.

In another particular example, the desired response is increasing cell proliferation, resulting in resulting in halting or slowing the progression of, or inducing a regression of a pathological condition or which is capable of relieving signs or symptoms caused by the condition. One example of a therapeutic effect is regression of the pathological condition, such as osteoporosis or heart disease, by inducing proliferation of bone cells or myocardial cells, respectively. Treatment can involve only slowing the progression of the disease temporarily, but can also include halting or reversing the progression of the disease permanently.

The therapeutically effective amount also includes a quantity of an agent (such as a Src protein, specific binding agent, or nucleic acid), sufficient to achieve a desired effect in a subject being treated. For instance, this can be the amount necessary to improve signs and/or symptoms a disease such as cancer, for example by decreasing cell proliferation of a Src-expressing tumor. Alternatively, this can be the amount necessary to improve signs and/or symptoms a disease such as heart disease or osteoporosis, for example by increasing cell proliferation of myocardial cells or osteogenic cells, respectively.

An effective amount of an agent (such as a Src protein, specific binding agent, or nucleic acid) can be administered in a single dose, or in several doses, for example daily, during a course of treatment. However, the effective amount of agent will be dependent on the source of agent applied (i.e. a protein isolated from a cellular extract versus a chemically synthesized and purified protein or a variant or fragment that may not retain fill activity), the subject being treated, the severity and type of the condition being treated, and the manner of administration. For example, a therapeutically effective amount of a Src protein, can vary from about 0.01 mg/kg body weight to about 1 g/kg body weight, such as about 1 mg per subject.

The methods disclosed herein have equal application in medical and veterinary settings.

Therefore, the general term “subject being treated” is understood to include all animals (e.g. humans, apes, dogs, cats, horses, and cows) that require modulation of a cell proliferation.

Therapeutically active molecule: An agent, such as an Src peptide (including variants, fusions, or fragments thereof) or Src specific binding agent, that can have a therapeutic effect, for example by modulating cell proliferation, as measured by clinical response (for example a decrease in tumor cell burden, or measurable reduction in the size of a tumor). Therapeutically active molecules can also be made from nucleic acids. Examples of nucleic acid based therapeutically active molecules are a nucleic acid sequence that encodes Src, wherein the nucleic acid sequence is operably linked to a control element such as a promoter. Therapeutically active agents can also include organic or other chemical compounds that mimic the effects of the peptide.

Transduced and Transformed: A virus or vector “transduces” or “transfects” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule might be introduced into such a cell including transfection with viral vectors, transformation with plasmid vectors, and introduction of naked DNA by electroporation, lipofection, and particle gun acceleration.

Transgene: An exogenous nucleic acid sequence supplied by a vector. In one example, a transgene encodes a Src polypeptide, such as SrcS17A or SrcS17D.

Tumor: A neoplasm. Includes solid and hematological (or liquid) tumors. In one example a tumor is a “Src-positive tumor” in which Src is overexpressed, as compared to Src expression in a same tissue type that is not neoplastic. Expression levels can be determined by immunocytochemistry. Examples of tumor types that overexpress Src are tumors of the breast, colon, pancreas, brain, lung, ovary, esophagus, and gastric tract, as well as melanoma, adenocarcinomas, retinoblastomas, neuroblastomas, and hematopoietic malignancies such as leukemias (Irby and Yeatman. Oncogene 19:563642, 2000).

Variants or fragments or fusion proteins: The disclosed Src proteins, and mutants thereof, include variants, fragments, and fusions thereof DNA sequences that encode for a protein, fusion protein, or a fragment or variant of a Src protein (for example a fragment or variant having at least 80%, 90% or 95% sequence identity to a Src protein), can be engineered to allow the protein to be expressed in eukaryotic cells, bacteria, insects, and/or plants. To obtain expression, the DNA sequence can be altered and operably linked to other regulatory sequences. The final product, which contains the regulatory sequences and the therapeutic protein, is referred to as a vector. This vector can be introduced into eukaryotic, bacteria, insect, and/or plant cells. Once inside the cell the vector allows the protein to be produced.

A fusion protein including a protein, such as Src (or variants, polymorphisms, mutants, or fragments thereof) linked to other amino acid sequences that do not inhibit the desired activity of Src, for example the ability to increase or decrease cell proliferation, depending on the phosphorylation state of Ser17. In one example, the other amino acid sequences are no more than 8, 9, 10, 12, 15, 20, 30, or 50 amino acid residues in length.

One of ordinary skill in the art will appreciate that the DNA can be altered in numerous ways without affecting the biological activity of the encoded protein. For example, PCR can be used to produce variations in the DNA sequence that encodes Src. Such variants can be variants optimized for codon preference in a host cell used to express the protein, or other sequence changes that facilitate expression.

Vector: A nucleic acid molecule as introduced into a host cell, thereby producing a transformed host cell. A vector may include nucleic acid sequences that permit it to replicate in the host cell, such as an origin of replication. A vector may also include one or more selectable marker genes and other genetic elements known in the art

### Specific-Binding Agents and Methods of Their Use

Specific-binding agents are disclosed that specifically bind to Src when Ser17 is phosphorylated, but do not bind to Src when Ser17 is not phosphorylated, as well as methods of using such agents as diagnostic agents. Examples of specific-binding agents include, but are not limited to, a drug or an antibody, such as a polyclonal antibody, a monoclonal antibody, a chimeric antibody, a humanized antibody, or fragments thereof. Also disclosed is an immortal cell line or hybridoma that produces a monoclonal antibody that specifically binds to Src when it is phosphorylated Ser17, but does not bind to Src when Ser17 is not phosphorylated.

Such specific-binding agents can bind to Src or any fragment, variant, polymorphismn, or mutant thereof, which includes phosphorylated Ser17. Examples of amino acid sequences that can be recognized by the specific-binding agents of the present disclosure include, but are not limited to: QRRRSLEPA (SEQ ID NO: 5), SQRRRSLEPAE (SEQ BD NO: 6), ASQRRRSLEPAEN (SEQ ID NO: 7), DASQRRRLEPAENV (SEQ ID NO: 8), KDASQRRRSLEPAENVH (SEQ ID NO: 9), PKDASQRRRSLEPAENVHG (SEQ ID NO: 10), and KPKDASQRRRSLEPAENVHGA (SEQ ID NO: 11)), wherein the underlined serine is phosphorylated. Such sequences can also be used to generate specific-binding agents, such as antibodies. One skilled in the art will understand that variants of these sequences can also be used, such as those that have greater or fewer amino acids, or those that have substituted amino acids, such as conservative substitutions, as long as the specific-binding agent retains the ability to specifically bind to Src when Ser17 is phosphorylated. In addition, fusion proteins that include these sequences can be used, as long as the specific-binding agent retains the ability to specifically bind to Src when Ser17 is phosphorylated.

A method is also disclosed for detecting Src when it is phosphorylated at Ser17, for example using the specific-binding agents discussed above, such as an antibody. The method includes contacting a sample with a specific-binding agent, under conditions that allow the specific-binding agent to specifically bind to Src when it is phosphorylated at Ser17. Upon binding, a specific-binding agent:Src phosphorylated at Ser17 complex is formed. The presence or absence of this complex is then detected, wherein the presence of the complex indicates that Src phosphorylated at Ser17 is present in the sample, which is associated with decreased cell proliferation. In one example, detection of the complex includes detecting a label on the specific-binding agent Examples of labels that can be used to practice the method include, but are not limited to, a radioisotope, a fluorophore, a bioluminescent compound, an enzyme, or a chemiluminescent compound. In one example, the method further includes quantitating the amount of the complex.

The sample can be any biological sample, such as a protein sample obtained from a tumor, wherein the absence of detectable specific-binding agent:Src phosphorylated at Ser17 complex in the sample is associated with increased cell proliferation. In one example, the tumor is a Src-positive tumor, such as a tumor of the breast, ovary, colon, brain, pancreas, lung, esophagus, skin, eye, hematopoietic system or gastric tract. In a more specific example, a Src-positive tumor is a melanoma, adenocarcinoma, retinoblastoma, or neuroblastoma.

### Method of Identifying Abnormal Proliferation

A method is disclosed for identifying a cell having abnormal proliferation, such as a cell with increased or decreased proliferation relative to a normal cell. The method includes detecting a mutation in Src at Ser17, wherein this mutation is associated with abnormal cellular proliferation. Any method known by those skilled in the art can be used to detect a Src mutation at Ser17. Examples of such methods include, but are not limited to, RT-PCR and PCR amplification of a Src nucleic acid sequence from the cell that includes a region encoding Ser17, hybridization with sequence-specific probes or agents such as antibodies, restriction digestion, and direct sequencing of Src.

In one example, the method identifies cells having increased proliferation by detecting a mutation in Src at Ser17 associated with increased cellular proliferation, such as a Src S17A mutation, that is associated with decreased phosphorylation at Src Ser17. In a particular example, the mutation is further associated with enhanced susceptibility of a subject to a Src-positive tumor. In another example, the method identifies cells having decreased proliferation by detecting a mutation in Src at Ser17 that is associated with mimicking phosphorylation of Src at Ser17, such as the S17D mutation, which is associated with decreased cellular proliferation.

### Methods of Screening Test Agents

A method is disclosed for screening one or more test agents to identify agents that alter cell proliferation. The method includes contacting one or more test agents, such as a test compound, drug, peptide, nucleic acid, antibody, or mimetic, with a cell in which Src is mutated at Ser17. The mutation at Ser17 alters Ser17 phosphorylation, thereby producing a cell having abnormal proliferation. An assay is performed to determine whether the test agents alters the abnormal cell proliferation. Examples of assays that can be used include, but are not limited to, a proliferation assay (such as the Mu assay disclosed herein) a Rap1 activation affinity assay, and a Ras 1 activation affinity assay.

In one example, the method is used to screen one or more test agents to identify agents that decrease cell proliferation by determining whether cell proliferation decreases in the presence of the test agent, when compared to cell proliferation in the absence of the test agent For example, the method can be used to identify agents that mimic Src when Src is phosphorylated at Ser17. Such agents will decrease cell proliferation. In particular examples, the mutation at Ser17 used to identify agents that decrease cell proliferation is an S17A mutation or an ablation of the Src gene (such as by using an SYF cell), where observed decreased proliferation in the presence of the test agent indicates that the test agent can compensate for Src mutations at Ser17 that decrease phosphorylation of Ser17.

Alternatively, the method is used to screen one or more test agents to identify agents that increase cell proliferation by determining whether cell proliferation increases in the presence of the test agent, when compared to cell proliferation in the absence of the test agent For example, the method can be used to identify agents that mimic Src when Src is not phosphorylated at Ser17. Such agents increase cell proliferation. In a particular example, the mutation at Ser17 used to identify agents that increase cell proliferation is an S17D mutation, where observed increased proliferation in the presence of the test agent indicates that the test compound can compensate for Src mutations at Ser17 that increase phosphorylation at Ser17, or mimic phosphorylation of Ser17.

A method of screening a test agent to identify agents that alter phosphorylation of Src at Ser17 is disclosed. The method includes contacting one or more test agents with a Src peptide that includes Ser17, generating a test sample. A kinase assay is performed on the test sample, and a determination is made as to whether there is enhanced Src phosphorylation at Ser17 compared to a test sample that does not include the test agent. Any kinase assay known in the art can be used.

In one example, the method is used to identify agents that increase phosphorylation of Src at Ser17, wherein phosphorylation of Src at Ser17 is associated with decreased cell proliferation. Alternatively, the method is used to identify agents that decrease phosphorylation of Src at Ser17, wherein dephosphorylation of Src at Ser17 is associated with increased cell proliferation.

### Methods of Altering Cell Proliferation

A method is disclosed for altering cell proliferation that includes altering phosphorylation of Src Ser17 in a cell, for example by contacting the cell with an agent that alters phosphorylation of Src Ser17. In one example, the method increases cell proliferation by decreasing phosphorylation of Src Ser17 in the cell, for example by expressing a Src S17A mutant in the cell, and/or by administration of a S17A mutant Src protein or peptide to the cell. Such a method can be used to grow or culture Src-positive cells ex-vivo, such as Src-positive tumor cells, for example breast cancer cells. In addition, such as method can be used to treat a disease in which increased cell proliferation is desired, such as heart disease, Alzheimer's, Parkinson's disease, and osteoporosis. Alternatively, the method decreases cell proliferation by increasing phosphorylation of Src Ser17 in the cell, or by mimicking phosphorylation of Src Ser17 in the cell. For example, an Src S17D mutant is expressed in the cell, and/or an S17D mutant Src protein or peptide is administered to the cell. Such a method can be used to treat a Src-positive tumor.

In one example, contacting the cell with an agent that alters phosphorylation of Src Ser17 includes administering an effective amount of the agent to a subject, such as a mammal, in whom altered cell proliferation is desired In one example, decreased cell proliferation is desired, and the agent promotes phosphorylation of Src Ser17. Examples of when decreased cell proliferation is desired include, but is not limited to when the subject has a Src-positive tumor, such as a tumor of the breast, ovary, colon, brain, eye, skin, pancreas, lung, esophagus, hematopoietic system, or gastric tract Alternatively, increased cell proliferation is desired, and the agent inhibits phosphorylation of Src Ser17. Examples of when increased cell proliferation is desired include, but are not limited to when the subject has heart disease, Alzheimer's, Parkinson's disease, or osteoporosis. Another example of when increased cell proliferation is desired is when the subject is a laboratory mammal, such as a primate, mouse, rat, or rabbit, in whom increased proliferation of a Src-positive tumor is desired.

Transgenic mammals are disclosed. Examples include transgenic humans and mice that include a mutation at Src Ser17 that alters phosphorylation of Src Ser17. In one example, the mutation at Src Ser17 inhibits phosphorylation of Src Ser17 and promotes cellular proliferation of cells in which the mutation is present. An example of such a mutation is an S17A Src mutation. In another example, the mutation at Src Ser17 promotes phosphorylation of Src Ser17, or mimics phosphorylation of Src Ser17, and inhibits cellular proliferation of cells in which the mutation is present. An example of such a mutation is a S17D Src mutation. Transgenic mammals having a mutation at Src Ser17 that alters phosphorylation of Src Ser17, can be used to screening for agents that affect cellular proliferation, by administering one or more test agents to the transgenic mammal and determining whether the agent offsets the altered phosphorylation of Src Ser17.

Disclosure of certain specific examples is not meant to exclude other embodiments.

### EXAMPLE 1

### C3G is Involved in cAMP/PKA Activation of Rap1

Forskolin is a potent activator of adenylyl cyclases that rapidly elevates intracellular cAMP levels. Forskolin's activation of endogenous Rap1 in NIH3T3 cells is blocked by pretreatment of cells with H89, a selective inhibitor of PKA, as well as the protein kinase inhibitor of PKA, PKI (Schmitt and Stork, Mol. Cell. Biol. 21:3671-83, 2001), demonstrating that cAMP's activation of Rap1 requires PKA in these cells. Although it is known that PKA phosphorylates Rap1 directly, the consequences of this phosphorylation were not clear.

To demonstrate that PKA activates Rap1 by phosphorylating proteins upstream of Rap1, the ability of lysates from Forskolin-treated cells to activate Rap1 within lysates prepared from unstimulated cells, was tested using the following protocol.

NIH3T3 cells (American Type Culture Collection, ATCC, Manassas, Va.) were cultured in Dulbecco-Modified Eagle Medium (DMEM) plus 10% fetal calf serum, penicillin/streptomycin, and L-glutamine at 37° C. in 5% CO₂. Cells were maintained in serum-free DMEM for 16 hours at 37° C. in 5% CO₂ prior to treatment. NIH3T3 cells were trasfected with Flag-Rap1 (see Vossler et al., Cell, 89:73-82, 1997) or stimulated with Forskolin (10 μM, Cal Biochem, Riverside, Calif.) for five minutes. Forskolin treated cells received either a pretreatment or post-treatment with 10 μM N-[2(Bromocimamylamino) ethyl]-5-isoquinolinesulfonamide (H89) (Cal Biochem, Riverside, Calif.) for 20 minutes.

Cells were co-transfected with Flag-Rap1 at seventy to eighty percent confluency with the cDNA using a Lipofectamine 2000 kit (Gibco BRL, Rockville, Md.) according to the manufacturer's instructions. The control vector, pcDNA3 (Invitrogen Corp.), was included in each set of transfections to assure that each plate received the same amount of DNA. Following transfection, cells were allowed to recover in serum containing media for 24 hours. Cells were then starved overnight in serum-free DMEM before treatment and lysis.

After treatment, cells were lysed in ice-cold lysis buffer (50 mM Tris-HCL (pH 8.0), 10% glycerol, 1% nonidet P40, 200 mM NaCl, 2.5 mM MgCl₂, 1 mM phenylmethylsulfonyl fluoride, 1 μM leupeptin, 10 μln soybean trypsin inhibitor, 10 mM NaF, 0.1 μM aprotinin, and 1 mM NaVO₄). Nuclei and cytoskeleton were removed from equivalent amounts of lysate by centrifugation at 5,000 rpm for five minutes. The supernatant was centrifuged at 100,000×g for one hour at 4° C. and, the resultant membrane was resuspended in cold lysis buffer. Aliquots of the membrane fraction were analyzed for the presence of C3G by Western blotting. The cytosolic or membrane fractions of treated or untreated cells were mixed with equivalent amounts of lysates from untreated Flag-Rap1 transfected cells. The mixture was incubated at 37° C. for five minutes and equal amounts of lysate were analyzed using the Rap1 activation assay to monitor Flag-Rap 1 activation by Gst-RalGDS.

The Rap1 activation affinity assay was performed as follows. A GST fusion protein of the Rap1-binding domain of RalGDS (GST-RalGDS, Dr. Bos, Utrecht University, The Netherlands) was expressed in E. coli following induction by isopropyl-1thio-β-D-galactopyranoside. Active Rap1 was isolated as previously described (Franke et al., EMBO J. 16:252-9, 1997). Briefly, equivalent amounts of supernatants (500 μg) were incubated with GST-RalGDS-Rap1 binding domain coupled to glutathione beads. Following a one hour incubation at 4° C., beads were pelleted and rinsed threes times with ice-cold lysis buffer. The protein was eluted from the beads using 2× Laemmli buffer and applied to a 12% SDS-polyacrylamide gel. Proteins were tansferred to PVDF membrane (Millipore Corporation, Bedford, Mass.), blocked in 5% milk for one hour and, probed with either α-Rap1/Krev-1 (Santa Cruz Biotechnology Inc., Santa Cruz, Calif.) or Flag antibody (Sigma, St Louis, Mo.) overnight at 4° C., followed by an HRP-conjugated anti-rabbit secondary antibody (or anti-mouse secondary for anti-Flag western blots). Proteins were detected using enhanced chemiluminescence.

Lysates from Forskolin-treated cells retained the ability to activate Flag-Rap1 expressed in untreated cells, and this activation in trans was blocked by pretreatment with H89, confirming a role for PKA. To determine if PKA activated Flag-Rap1 directly, H89 was added ten minutes after the addition of Forskolin to permit PKA-dependent phosphorylation of substrates to occur prior to mixing of Forskolin-treated and untreated Flag-Rap1-expressing lysates. Lysates from cells in which H89 was applied after Forskolin were still able to activate Flag-Rap1 in trans. Since this protocol ensures that PKA cannot phosphorylate Flag-Rap1 directly, these results demonstrate that PKA can activate Rap1 via proteins upstream of Rap1.

To determine whether the proteins that activate Rap1 reside within the membrane or cytosolic components of the treated cells, NIH3T3 cells were transfected with Flag-Rap1 or stimulated with Forskolin and pre-treated with H89 as described above. The cytosolic or membrane fractions from treated lysates were prepared and incubated with Flag-Rap1 lysates and analyzed for Flag-Rap1 activation as described above. Only the membrane fraction of Forskolin-treated NIH3T3 cells activated Rap1 in trans, and PKA was needed.

To determine the role of the Rap 1-GEF, C3G, in cAMP activation of Rap1, C3G was immunodepleted from the membrane fraction. This C3G-depleted fraction was used to determine whether this would limit Forskolin's activation of Rap1. The membrane fractions of untreated or Forskolin treated NIH313 cells, generated as described above, were resuspended in equal volumes of lysis buffer. Immunoprecipitating antibodies (C3G, SOS, or ERK2 antibodies, 10 μl of a 200 μg/ml stock, all from Santa Cruz Biotechnology Inc.) were added to the membrane fraction and immunoprecipitated six hours at 4° C. Following the immunoprecipitation, the precipitates were cleared from the tubes and analyzed by Western blotting for C3G. The remaining membrane components were incubated at 37° C. for five minutes with equal amounts of Flag-Rap1 lysate and subjected to the Rap 1 activation assay as described above or analyzed by Western blotting for C3G.

Immunodepletion with antibodies to C3G, but not control antibodies SOS and ERK2, eliminated C3G from the membrane. Membranes immunodepleted of C3G could no longer activate Rap1 in trans, whereas immunodepletion of the related Ras exchanger, SOS, did not block Forskolin's effects. Immunodepletion of ERK2 served a negative control. These results demonstrate a role of C3G in PKA activation of Rap1.

To determine if C3G is recruited to membranes upon its activation, NIH3T3 cells were untreated or stimulated with Forskolin in the presence or absence of H89 as described above. Following lysis of the cells, the resulting membrane fraction was analyzed by Western blot for the presence of C3G. Total cell lysates were also examined for the presence of C3G. It was observed that C3G rapidly moved into the membranes following Forskolin stimulation, and this was PKA dependent. Therefore, PKA activation of Rap1 is indirect and involves a component of the membrane fraction of treated cells, C3G.

### EXAMPLE 2

### The Role of Crk-L, CbI, and Src in cAMP/PKA Activation of Rap1

C3G exists in the cytoplasm in a complex with a small adaptor molecules including Crk-L, Crk-I, and CrkII (Knudsen et al., J. Biol. Chem. 269:32767, 1994; Tanaka et al., Proc. Natl. Acad. Sci. USA 91:3443-7, 1994). Upon stimulation by growth factors, the Crk/C3G complex is recruited to the membrane where it binds to scaffolding molecules including FRS2 (Kao et al., J. Biol. Chem 13:13, 2001), IRS-1 (Beitner-Johnson et al., J. Biol. Chem. 271:9287-90, 1996; Sorokin et al., Oncogene, 16:2425-34, 1998), and Cb1 (Reedquist et al., J. Biol. Chem. 271:843542, 1996; Xing et al., Mol. Cell. Biol. 20:7363-77,2000), which are tyrosine phosphorylated following growth factor stimulation and bind Crk upon phosphorylation via Crk's SH2 domain. Multiple scaffold molecules are expressed in NIH3T3 cells including Cb1 and IRS-1 (Broome et al., Oncogeyie 18:2908-12, 1999), but IRS-1 is not tyrosine phosphorylated by Forskolin in these cells (Calleja et al., Endocrinology 138:1111-20, 1997).

To determine the role of C3G and its complex in Rap1 activation, NIH3T3 cells were grown and treated for 20 minutes in the presence or absence of H89 (10 μM) or PP2 [AG1879; 4-amino-5-(4-chlorophenyl)-7-(t-butyl)pyazolo[3,4-d]pyrimidine, LY294002 [2-(4-Morpholinyl)-8-phenyl-4H-1-bemzopyran-4-one] (10 μM, Cal Biochem), treated with Forskolin (10 μM, Cal Biochem) for five minutes, and cell lysates prepared, as described above. Endogenous Cb1 was immunoprecipitated using a Cb1 antibody (Santa Cruz Biotechnology Inc.) from cell lysates and analyzed by Western blot for tyrosine phosphorylation, using a pTyr antibody (Santa Cruz Biotechnology Inc.) as described above. Briefly, for immunoprecipitation of Cb1, equal amounts of cell lysate per condition were precipitated at 4° C. for 4 to 6 hours in lysis buffer. Proteins were resolved by SDS-PAGE, blotted onto PVDF membranes and probed with the appropriate antibodies. All Western blots and immunoprecipitations were performed at least three times.

Endogenous Cb1 was tyrosine phosphorylated following Forskolin stimulation of NIH3T3 cells. This phosphorylation was inhibited by both H89 and PP2, a selective inhibitor of Src family kinases (SFKs), indicating that both PKA and SFKs are involved in Cb1 phosphorylation.

To determine if Cb1 forms part of a complex, cells were stimulated with Forskolin in the presence or absence of H89 or PP2 and lysates generated as described above. Myc-Cb1 was immunoprecipitated from treated cells and the pellets analyzed by Western blot using antibodies specific for Crk-L and C3G. Total cell lysates were examined for myc-Cb1 by Western blot using a myc antibody (Santa Cruz Biotechnology Inc.). NIH3T3 cells express Crk-L, an isoform of Crk that is constitutively bound to C3G. Forskolin treatment of NIH3T3 cells stimulated the formation of a complex including C3G, Crk-L, and CbL Formation of this complex was blocked by both H89 and PP2.

To determine whether the Cb1/CrkIC3G complex was involved in Rap1 activation by PKA, NIH3T3 cells were transfected using Lipofectamine (see EXAMPLE 1), with Flag-Rap1 and one of the following mutants: CBR, a truncated form of C3G that interferes with Crk function (see York et al., Nature, 392:622-6, 1998); Cb1-ct; a carboxyl-terminal fragment of Cb1 that blocks Cb1 function (Brian Druker, OHSU, Portland, Oreg.), or a kinase-dead Src mutant (SrcK296R, or dSrc, Karin Rodland, OHSU, Portland, Oreg.). Lysates were prepared and subsequently analyzed for Rap1 activation as described in EXAMPLE 1. Rap1 activation by Forskolin was blocked by each interfering mutant.

Flag-Rap1 was co-transfected with constitutively active Src (SrcY527F, or c.a.Src, Karin Rodland, OHSU) into NIH3T3 cells using Lipofectamine (see EXAMPLE 1), along with CBR and Cb1-ct Cells lysates were then analyzed for Rap1 activation (Flag-Rap1-GTP). Constitutively active Src is sufficient to activate Rap1. Both C3G/Crk and Cb1 were also required for activation of Rap1. Thus, cAMP's activation of Rap1 involves the recruitment of C3G/Crk to a membrane-associated complex with Cb1, and Src is both necessary and sufficient for this action.

To determine whether this pathway was shared by hormones and agonists of G protein-coupled receptors (GPCRs) known to be linked to Gsa and cAMP, the following methods were used. Isoproterenol, an agonist of the p2-adrenergic receptor, activates Rap1 (Schmitt and Stork, J. Biol. Chem. 275:25342-50, 2000). NIH3T3 cells were left untreated or treated with isoproterenol (10 μM, Sigma) or prostaglandin E (PGE₁, 10 μM, Sigma) for five minutes in the presence or absence of either H89 or PP2 as described above. Lysates were then analyzed for activation of endogenous Rap1 as described in EXAMPLE 1. Both isoproterenol and PGE, activated Rap1 in NIH3T3 cells via PKA and SFKs. This demonstrates that Forskolin and hormonal elevation of cAMP use similar mechanisms to activate Rap1.

The results disclosed herein demonstrate that the positive regulation of Rap1 by PKA is indirect, and involves the Rap1 activator C3G. In addition, cAMP's activation of PKA triggers the recruitment of Crk-C3G complexes to the scaffold protein Cb1. These results indicate that the Cb1/Crk/C3G complex is assembled following stimulation of cells by both cAMP-linked hormones and growth factors. However, Src activation of Cb1/Crk/C3G can be triggered by pathways other that those initiated by cAND/PKA. For example, Src-family kinases may phosphorylate specific sites in Cb1 to initiate binding of Crk. These results are consistent with previous reports defining Cb1 as a negative regulator of ERK signaling (Rellahaa et al., J. Bio. Chem. 272:30806-11, 1997) and cell growth (Broome et al., Oncogene 18:2908-12, 1999; Murphy et al., Mol. Cell. Biol. 18:4872-82, 1998; Thien and Langdon, Nat. Rev. Mol. Cell. Biol. 2:294307, 2001). Although Cb1-mediated ubiquination and degradation of mitogenic signaling contributes to these actions, the results disclosed here indicate that Rap1 is an additional anti-proliferative target.

### EXAMPLE 3

### The Role of Src in PKA Inhibition of ERKs and Cell Proliferation

The antagonism of growth factor signaling by Rap1 is thought to be due to Rap1 's ability to sequester Raf-1 away from Ras. In NIH3T3 cells, it has previously been shown that cAMP/PKA triggers the association of Rap1 and Raf-1, and that this requires active Rap1 (Schmitt and Stork, Mol. Cell Biol. 21:3671-83, 2001).

NIH3T3 cells were transfected using Lipofectamine (see EXAMPLE 1) with polyhistidine-tagged Rap1 along with CBR or Cb1-ct (see EXAMPLE 2) as previously described (Schmitt and Stork, Mol. Cell. Biol. 21:3671-83, 2001; Schmitt and Stork, J. Biol. Chem. 275:25342-50,2000). Cells were left untreated or stimulated with Forskolin, as described in EXAMPLE 1. Subsequently, cells were lysed in ice-cold buffer containing 1% NP40, 10 mM Tris, pH 8.0, 20 mM NaCl, 30 mM MgCl₂, 1 mM PMSF, and 0.5 mg/ml aprotinin and supernatants prepared by low speed centrifugation. Transfected His-Rap1 proteins were precipitated from supernatants containing equal amounts of protein using Ni-NTA agarose (Qiagen Inc., Chatswoth, Calif.) and washed with 20 mM imidazole in lysis buffer and eluted with 500 mM imidazole and 5 mM EDTA in phosphate-buffered saline. The eluates containing His-Rap1 proteins were separated on SDS-PAGE, blotted onto PVDF membranes, and Raf-1 proteins detected by Western blotting using anti-Raf-1 antibodies (Santa Cruz Biotechnology Inc.).

It was shown that the association between Raf-1 and Rap1 was blocked by CBR, Cb1-ct, and PP2 indicating that sequestration of Raf-1 by Rap1, as well as Rap1 activation itself, requires C3G, Crk, Cb1, and SFKs.

To determine the role of SFKs in cAW's antagonism of extracellular signal regulated kinase (ERK) activation, the following methods were used. NIH3T3 cells were incubated in the presence or absence of either H89 or PP2 for 20 minutes, pretreated with Forskolin for five minutes, and then stimulated with 100 ng/ml, epidermal growth factor (EGF, Sigma), or 100 ng/ml platelet-derived growth factor (PDGF, Sigma) for five minutes, as described in EXAMPLE 1. Cell lysates were analyzed by Western blot for phosphorylation of endogenous ERK1/2 or total ERK2. To detect phosphorylation of endogenous ERK1/2, phosphorylation-specific ERK antibodies (PERK) that recognize phosphorylated ERK1 (pERK1) and ERK2 (pERK2), at residues threonine 183 and tyrosine 185 were used (New England Biolabs, Beverly, Mass.). Forskolin blocked both EGF- and PDGF-mediated ERK activation in NIH3T3 cells. Although inhibition of SFKs had no observable effect on either EGF's or PDGF's activation of ERKs, it prevented Forskolin's inhibition of ERKs.

To directly examine the role of Src in cAMP's activation of Rap1, mouse embryonic fibroblasts in which the genes encoding Src, Yes, and Fyn are ablated (SYF) (Klinghoffer et al., EMBO J. 18:2459-71, 1999) were used. As a control, fibroblasts ablated for Yes and Fyn, but remained wild type at the Src locus (Src⁺⁺ were used (Klinghoffer et al., EMBO J. 18:2459-71, L999). It has been previously shown that PDGF stimulation of ERKs and proliferation does not require Src family kinases in SYF cells (Klinghoffer et al., EMBO J. 18:2459-71, 1999). Therefore, this model system is well suited to examine Src's potential anti-proliferative role in cAMP signaling.

Src⁺⁺ or SYF cells, obtained from ATCC, were cultured as described in EXAMPLE 1 for NIH3T3 cells. Cells were untreated or stimulated with Forskolin and/or PDGF, as described above. Lysates were analyzed by Western blot for activation of either endogenous ERK1/2 (PERK, top panel), or Rap1 (Rap1-GTP, bottom panel) as described above. cAMP inhibited PDGF-mediated ERK activation in Src⁺⁺ cells, but not in SYF cells. Moreover, although cAMP robustly activated Rap1 in Src⁺⁺ cells, cAMP did not activate Rap1 in SYF cells.

To confirm this result, SYF cells were transfected with wild type Flag-Src or the pMACS 14.1 control vector along with pMACS K^{k}.II positive selection plasmid as specified by the manufacturers guidelines (Miltenyi Biotec). The wild-type Flag-Src construct was generated following subcloning wild-type Src cDNA (Upstate Biotechnology, Lake Placid, N.Y.) into Bluescript KS (Clonetech, Palo Alto, Calif.). The N-terminal half of Src was then cut with Hind m and an internal BspHI site. The C-terminal half of Src was generated by PCR from wild type Src, using specific primers to the sequence (sense oligo: ATGTCCCCAGAGGCCTTCCTGCAGGAC, SEQ ID NO: 3; and antisense oligo: TTAAATCCTAGGTTCTCCCCGGGCTCGTACTGTGGCTCAGTGGA, SEQ ID NO: 4) and then digested with BspHI and BamHI and subcloned with the N-terminal fragment of Src into pcDNA3 containing a 2× C-terminal Flag. Cells were co-transfected with Flag-Rap1 at 70-80% confluency with the cDNA using Lipofectamine (as described in EXAMPLE 1). Following the selection of SYF cells, cells were eluted in DMEM plus 10% fetal calf serum and recovered for 24 hours on 10 cm plates.

Following selection, SYF cells were left untreated or stimulated with Forskolin in the presence or absence of H89 or PP2. Cells were lysed in ice-cold lysis buffer and the lysates analyzed by Western blot for phosphorylation of myc-ERK2 as described above. The lysates were also probed with antibodies to ERK2 as a loading and transfection control (transfected myc-ERK2 migrated just above endogenous ERK2). In addition, the cell lysates were examined for Rap1 activation using the method described in EXAMPLE 1. Similar responses to cAMP (activation of Rap1, inhibition of ERKs) were observed in SYF cells transfected with wild-type Src. These results demonstrate that Src-mediates cAMP's inhibition of ERK via Rap1 activation To determine if other members of the SFK could mediate cAMP's activation of Rap1, SYF cells were transfected with Flag-Rap1 and cDNAs encoding wild type Src, Yes, Fyn, or Lck. Wild type Fyn, Lck, and Yes (Andrey Shaw, Washington University, St. Louis, Mo.) were subcloned into pcDNA3 and transfected into cells using Lipofectamine (see EXAMPLES 1 and 3). Subsequently, the cells were stimulated with Forskolin, and the cell lysates were examined for Rap1 activation using the methods described in EXAMPLE 1. The ability of Src to mediate cAMP's activation of Rap1 was not shared by related members of the SFK family including Yes, Fyn, and Lck, indicating that this action of Src is unique among SFKs.

The scaffolding protein Cb1 appeared to be involved in PKA activation of Rap1 in NIH3T3 cells. Therefore, to determine whether Src was involved in the PKA-dependent phosphorylation of Cb1 in mouse embryonic fibroblasts, the following method was used. Src⁺⁺ or SYF cells were treated with Forskohn in the presence or absence of H89 or PP2 as described in EXAMPLE 1. In some samples, SYF cells were transfected with myc-Cb1 and Src using the transfection methods described above. Cb1 was immunoprecipitated from cell lysates and analyzed by Western blot for tyrosine phosphorylation of Cb1or total Cb1 protein Similar to the result observed in NIH3T3 cells, Forskolin stimulated the tyrosine phosphorylation of Cb1 in a PKA- and Src-dependent manner in Src⁺⁺ +cells, as well as SYF cells transfected with wild-type Src, but did not stimulate Cb1 phosphorylation in untransfected SYF cells.

To determine the effect of Src on cell proliferation, the following protocol was used. Src⁺⁺ and SYF cells were serum-starved overnight and plated onto 96 well plates. Cells were then treated with PDGF, EGF, Forskolin, H89, and/or PD98059 (PD, 10 μM, Cal Biochem). Cells were analyzed 48 hours later by MTT assay and data quantified (n=4±S.E.). The MTT assay for cell proliferation was conducted as follows (Schmitt and Stork, Mol. Cell. Biol. 21:3671-83, 2001). Briefly, 2.5 hours prior to lysis, 20 μl of sterile 2.5 μg/ml MTT (Sigma) was added to the cells and allowed to incubate at 37° C. At the appropriate time, cells were lysed and proteins solubilized in 50% volume/volume H₂O and N,N,-dimethylformamide containing 20% SDS, 0.5% of 80% acetic acid, and 0.4% 1M HCL Plates were read using a microplate reader and presented as the difference between optical densities at 570 and 650 mm.

PDGF stimulation of proliferation was dependent on ERK signaling in both Src⁺⁺ and SYF cells, as shown by experiments using the selective MEK inhibitor PD98059, which is similar to previous results examining EGF's action in NIH3T3 cells (Schmitt and Stork, Mol. Cell Biol. 21:3671-83, 2001). Forskolin treatment of Src⁺⁺ cells inhibited this response in a PKA-dependent manner. However, in SYF cells, Forskolin was unable to inhibit growth factor stimulation of cell growth. These data demonstrate for the first time the role of Src in cAMP's anti-proliferative actions.

### EXAMPLE 4

### Activation of Src by PKA Phosphorylation of Serine 17 (Ser17)

To examine the mechanism of cAMP regulation of Src, Src kinase activity was assayed in vivo by monitoring autophosphorylation of Src at tyrosine 416 (Tyr416) using previously disclosed methods (Brown and Cooper, Biochim. Biophys. Acta 1287:121-49, 1996; Martin, Nat. Rev. Mol. Cell. Biol. 2:467-75,2001).

Briefly, Src⁺⁺ and SYF cells transfected with wild type Flag-Src, were cultured and treated with Forskolin in the presence or absence of H89 or P22 as described in the above examples. Cell lysates were prepared, and Src immunopreciptiated and analyzed by Western blot for either tyrosine phosphorylation at site 416 (p416Src) using phospho-Src (Tyr416) antibodies (Cell Signaling Technology), Flag-Src, or analyzed by Western blot for either PKA phosphorylation or Src (using Src antibodies, Santa Cruz Biotechnology Inc.), using the methods described above. Alternatively, Flag-Src was immunoprecipitated from the cell lysates and analyzed by Western blot for either Cb1 or Flag-Src.

Forskolin stimulated phosphorylation of Src at tyrosine 416 (Tyr416) in both endogenous Src and transfected Flag-Src by the kinase activities of both PKA and Src. Activated Src associates with and directly phosphorylates Cb1 in vivo (Thien and Langdon, Nat. Rev. Mol. Cell. Biol. 2:294-307, 2001). Forskolin treatment induced an association between Src and Cb1 in Flag-Src-transfected SYF cells that was also dependent on the kinase activities of both PKA and Src. These data indicate that PKA stimulates Src kinase activity as measured by Src's autophosphorylation and association/phosphorylation with one of its endogenous substrates, Cb1.

Src contains one consensus PKA site at serine 17 (Ser17) within its N-terminus that represents the major site of serine phosphorylation within Src (Brown and Cooper, Biochim. Biophys. Acta 1287:12149, 1996). To further demonstrate that PKA phosphorylates endogenous Src in vivo, an antibody designed to recognize phospho-serine/threonine residues preceded by arginine at the −3 position (RXXpS/T), comprising the recognition site of PKA as well as other arginine-directed kinases not activated by Forskolin, was used (Cell Signaling Technology). The ability of this antibody to recognize Src was increased by Forskolin treatment, in PKA kinase activity dependent manner. Using this antibody, the ability of PKA to phosphorylate Ser17 directly was determined as follows.

A peptide identical to amino acids 9-25 within the N-terminus of wild-type Src (WT) (KDASQRRRSLEPAENVH; SEQ ID NO: 12), which contains the potential PKA phosphorylation site (underlined), was generated using an Auto-Spot Robot ASP 222 (ABiMED, Langenfeld, Germany) and spotted onto a nitrocellulose membrane. Control peptides with serines 12 and/or 17 replaced by alanine (S12A, SEQ ID NO: 13; S17A, SEQ ID NO: 14; and S12AS17A, SEQ ID NO: 15), serine 17 replaced by aspartate (S17D, SEQ ID NO: 16), and arginines at 14-16 replaced by alanine (R14A, SEQ ID NO: 17;
R15A, SEQ ID NO: 18; R16A, SEQ ID NO: 19), as well as peptides designed to both a scrambled (SEQ ID NO: 20) and an unrelated (FLAG) sequence (SEQ ID NO: 21), were also generated and spotted onto the membrane.

Using a method previously described (Tegge et al., Biochen. 34:10569-77, 1995), the membranes were pre-incubated at room temperature (RT) overnight (ON) in buffer containing 20 mM Hepes, pH 7.4, 100 mM NaCl, 2 mM MgCl₂, 1 mM EDTA, 1 mM DTI, and 0.2 mg/ml BSA. Membranes were then blocked at 30° C. for one hour in buffer containing 20 mM Hepes, pH 7.4, 100 mM NaCl, 2 mM MgCl₂, 1 mM EDTA, 1 mM DTT, and 1 mg/ml BSA and 30 μM cold ATP. To perform the kinase assay, the membranes were incubated at 30° C. for 30 minutes in buffer containing 20 mM Hepes, pH 7.4, 100 mM NaCl, 2 mM MgCl₂, 1 mM EDTA, 1 mM DTT, and 0.2 mg/ml BSA, and 12.5 nM purified catalytic subunit of PKA (John Scott, OHSU, Vollum Institute, Portland, Oreg.). Membranes were washed at R_{T} 10×10 minutes in 1M NaCl, 3×5 minutes in ddH₂O, 3×10 minutes in 5% H3PO₄, and 3×5 in ddH₂O. The membranes were then blocked, probed, and analyzed using chemiluminescence according the manufacturers guidelines for the phospho-PKA substrate antibody. Only the peptides containing Ser17 and adjacent arginines (WT and S12A) were recognized by the antibody, consistent with the consensus PKA recognition motif.

To determine if Ser17 is necessary for PKA phosphorylation of Src in vivo, a cDNA encoding a Src protein that had its serine at residue 17 substituted by an alanine (Flag-SrcS17A) was transfected into SYF cells. The Src S17A mutant was generated by PCR site directed mutagenesis. Coding regions of all plasmids were sequenced in both directions prior to transfection. SYF cells were transfected with either Flag-Src or Flag-SrcS17A using Lipofectamine as described in EXAMPLES 1 and 3. The transfected cells were stimulated with Forskolin for five minutes, then stimulated with PDGF or EGF for five minutes, using the methods described in the examples above. Cells were lysed in ice-cold lysis buffer as described in EXAMPLE 1, and 500 μg of cell protein was incubated with Flag antibody for immunoprecipitation of Flag-Src. The resulting immunoprecipitate was analyzed by Western blot for either PKA phosphorylation of Src or the presence of Flag-Src. Following transfection and Forskolin treatment, phosphorylation of wild type Src, but not Flag-SrcS17A, was detected, confirming that Ser17 was the major PKA phosphorylation site located within Src that was recognized by this antibody. Therefore, Src is primarily phosphorylated both in vitro and in vivo at Ser17 by PKA, but not by EGF or PDGF.

The activation of wild type Src by Forskolin was confirmed by observing autophosphorylation of Src at Tyr416 and using an in vitro kinase assay. To measure autophosphorylation of Src at Tyr416, SYF cells were transfected with either Flag-Src (WT) or Flag-SrcS17A and treated with Forskolin and PDGF or EGF, as described above. Flag-Src was immunoprecipitated from cell lysates and analyzed by Western blot for either phosphorylation of Src on Tyr416 or the presence of Flag-Src.

The in vitro kinase assay was performed as follows. SYF cells were transfected with Flag Src, Flag-SrcS17A, or Flag-SrcS17D and treated with Forskolin as described above. Cells were lysed and 500 μg of cell protein was incubated with Flag antibody for immunoprecipitation using the methods described in the above examples. Following immunoprecipitation and washing of Flag proteins, samples were subjected to an in vitro protein tyrosine kinase assay according to the manufacturer's guidelines (Life Technologies-Invitrogen, Carlsbad, Calif.). The assay utilizes a peptide substrate (RR-Src) specific for tyrosine kinases. Briefly, immunoprecipitated proteins were reconstituted in 10 μl of buffer and incubated with 10 μl of either 2× substrate solution or 2×control solution both containing 0.5 μCi [γ-³²P]ATP. The reaction mixture was incubated at 30° C. for 30 minutes and the reaction was stopped by the addition of 20 μl of ice cold 10% trichloroacetic acid. Following a 10-minute centrifugation at 4° C. and 14,000 rpm, 20 μl of supernatant from each reaction was spotted onto individual phosphocellulose discs. Discs were washed 2× in 1% acetic acid, placed into scintillation vials, and counted using a scintillation counter. Specific activity incorporated into peptide was calculated according to the manufacturer's guidelines and presented as counts per minute (cpm) (n=3+S.D.).

Forskoliu was unable to activate the SrcS17A mutant in either assay. A Src mutant containing aspartate at position 17 (SrcS17D) showed elevated (>2-fold) kinase activity.

To determine if constitutive phosphorylation on Tyr416 would be increased by Forskolin treatment, SYF cells were transfected with Flag-Src or Flag-SrcS17D, treated with Forskolin, lysed, proteins immunoprecipitated using the Flag antibody and analyzed by Western blot for phosphorylation of Src on tyrosine 416 or the presence of the Flag epitope. It was observed that constitutive phosphorylation on Tyr416 was not further increased by Forskolin treatment.

To determine if the SrcS17D would constitutively activate Rap1, SYF cells were co-transfected with Flag-Rap1 and either pcDNA3 vector DNA or Flag-Src and treated with Forskolin, or transfected with Flag-SrcS17D as indicated. Lysates were then analyzed for Rap1 activation as described in EXAMPLE 1 or analyzed by Western blot to control for levels of Flag-containing proteins. SYF cells expressing SrcS17D showed constitutive Rap1 activation.

SYF cells were transfected with mycERK2 and either vector DNA, Flag-Src, or Flag-SrcS17D and treated with PDGF plus or minus Forskolin, as described above. ERK phosphorylation was assayed following myc immunoprecipitation and western blotting. The lysates were also probed with antibodies to ERK2 (mycERK2) as a loading and transfection control. SYF cells expressing SrcS17D lacked ERK activation in response to PDGF. This is consistent with the mutant aspartate residue mimicking serine phosphorylation at this site. These data indicate that PKA activation of Src in vivo involves Ser17, and this site specifically mediates cAMP's activation of Src. EGF and PDGF activated both wild type Src and SrcS17A equally, indicating that the SrcS17A mutant retained proper protein folding for activation by growth factors and catalytic activity.

### EXAMPLE 5

### Src Ser17 Phosphorylation is Involved in Rap1 Activation and ERK Inhibition by PEA

To determine the role of Src Ser17 phosphorylation in cAMP activation of Rap1, Rap1 activation was assayed in SYF cells expressing a mutant Src protein (Flag-SrcS17A).

SYF cells were co-transfected with Flag-Rap1 and either vector, Flag-Src, or Flag-SrcS 17A and stimulated with Forskolin as described in the above examples. Cell lysates were examined for Rap1 activation using the method described in EXAMPLE 1 or analyzed by Western blot to identify Flag-containing proteins. The Flag antibody detects both Flag-Src and Flag-Rap1. In contrast to wild type Flag-Src, Flag-SrcS17A was not able to reconstitute Forskolin's activation of Rap1, indicating that that PKA phosphorylation of Ser17 on Src was required for Rap1 activation.

To determine the ability of other Src mutants to activate Rap1, Src4 cells were co-transfected with Flag-Rap1 and either CBR, Cb1-ct, dCnSrc, or SrcS17A and stimulated with Forskolin using the methods described in the above examples. Cell lysates were examined for Rap1 activation or Flag-Rap1. Expression of SrcS17A inhibited the ability of Forskolin to stimulate Rap1 activation in Srct+, despite the presence of endogenous wild type Src in these cells. This indicates that overexpression of the mutant interfered with the function of endogenous Src to mediate cAMP's action. Similar to the results observed in NIH3T3 cells, d.n.Src, Cb1-ct, and CBR all blocked Forskolin's actions in Srcw cells.

The role of Ser17 in Forskolin's ability to inhibit PDGF's activation of ERK was determined using the following methods. SYF cells were co-transfected with myc-ERK2 and either vector, Flag-Src, or Flag-SrcS17A and stimulated with Forskolin and PDGF, alone or in combination as described above. Myc-containing proteins were immunoprecipitated from cell lysates and analyzed by Western blot for ERK phosphorylation or myc. Only wild type Src, but not Src17A, restored Forskolin's inhibition of ERK in SYF cells.

To demonstrate that Flag-SrcS17A could block cAMP's inhibition of cell growth, SYF cells were transfected with Flag-Src or Flag-SrcS17A, then treated with Forskolin, PDGF, and/or EGF, alone or following pretreatment with PD98059 (PD) or H89 as described in the above examples. To quantitate cell proliferation, cells were analyzed 48 hours later by MTT assay (see EXAMPLE 3) and data was quantified (n=4±S.E.). PDGF and EGF both stimulated SYF cell growth in an ERK-dependent manner that was blocked by PD98059. Expression of Flag-Src in SYF cells restored Forskolin's ability to inhibit PDGF- and EGF-mediated cell growth. In contrast, expression of Flag-SrcS17A in these cells was not able to restore Forskolin's ability to inhibit either PDGF or EGF-stimulated cell growth.

Therefore, phosphorylation of Src Ser17 is a primary target of PKA and plays a role in the ability of cAMP to activate both Src and Rap1, as well as to antagonize growth factor signaling to both ERKs and cell growth.

### EXAMPLE 6

### Isoproterenol Activation of Ras and Rap1

In Hek293 cells, the β2-adrenergic receptor agonist isoproterenol stimulates endogenous receptors to activate ERKs through a PKA-dependent pathway. Isoproterenol's activation of PKA induces activation of Ras via a Src-dependent mechanism that is mediated by Gpy subunits. However, isoproterenol and PKA can also activate Rap1 and ERKs in Hek293 cells. As described in the above examples, in NIH3T3 cells, PKA activation of Rap1 results from the direct phosphorylation by PKA on the Src tyrosine kinase. In NIH3T3 cells that do not express B-Raf, PKA and Rap1 antagonize Ras-dependent activation of ERKs (Chen and Iyengar, Science 263:1278-81, 1994; Schmitt and Stork, Mol. Cell. Biol. 21:3671-83, 2001). In addition to antagonizing Ras, Rap1 can activate ERKs in cells that express the MAP kinase B-Raf (Ohtsuka et al. J. Biol. Chem. 271:1258-61, 1996). However, the contribution of Src in this action of Rap1 was previously unknown. Since isoproterenol couples efficiently to both Ras and Rap1 in Hek293 cells, this model system was used to determine the requirement of Src in each process.

Hek293 cells (ATCC) were cultured in DMEM plus 10% fetal calf serum, penicillin/streptomycin, and L-glutamine at 37° C. in 5% CO₂. Cells were maintained in serum-free DMEM for 16 hours at 37° C. in 5% CO₂ prior to treatment with H89 (10 μM) or PP2 (10 μM) for 20 minutes, and then treated with isoproterenol (10 μM) for 5 minutes. Cell lysates were prepared, and a Rap1 activation affinity assay performed as described in Example 1. In Hek293 cells, isoproterenol activated endogenous Rap1 in a PKA-dependent manner. This activation required Src family kinases (SFKs) as it was blocked by the inhibitor PP2.

Endogenous GTP-loaded Ras was determined using a Ras activation affinity assay as follows. Hek293 cells were grown, stimulated, and lysed as described above. Activated Ras was assayed as previously described (Schmitt and Stork, Mol. Cell. Biol. 21:3671-83, 2001). Briefly, equivalent amounts of lysates from stimulated cells were incubated with GST-Rafl-RBD (Ras-binding domain) as specified by the manufacturer (Upstate Biotechnology, Lake Placid, N.Y.). Proteins were eluted with 2×Laemmli buffer and applied to a 12% SDS polyacrylamide gel. Proteins were transferred to a PVDF membrane, blocked at room temperature for 1 hour in 5% milk and probed with either Ras or Flag antibody overnight at 4° C., followed by an HRP-conjugated anti-mouse secondary. Proteins were detected using enhanced chemiluminescence. It was observed that isoproterenol activated endogenous Ras via SFKs, but this proceeded through a PKA-independent pathway.

### EXAMPLE 7

### Role of Src Kinase in Rap1 and Ras Activation by Isoproterenol

To determine which SFK was mediating these effects, cell lines derived from mouse embryo fibroblasts that lack SFKs, were used as described in Example 3. SYF and Srcd cells were transfected with Flag-Rap 1, and WT Src or pcDNA3 vector alone as described in Example 3. Cells were stimulated with isoproterenol or left untreated, and then cell lysates were examined for active GTP-loaded Rap1 (Flag-Rap1-GTP) as described in Examples 1 and 6. Isoproterenol's ability to activate Rap1 was absent in SYF cells, but was retained in Src⁺⁺ cells. Similarly, isoproterenol's ability to activate Ras was also absent in SYF cells, but retained in Src4 cells. Additionally, both actions of isoproterenol were reconstituted by transfecting wild type Src into SYF cells. Taken together, these data demonstrate that Src is required for isoproterenol's activation of both Ras and Rap1 in these cells.

The ability of cAMP to activate Rap1 in selected cell types was shown in the above examples to require Src and PKA, through the PKA-dependent phosphorylation of Src at serine 17 (S17). To address the requirement of S17 phosphorylation in Ras and Rap1 signaling, activation by isoproterenol in cells expressing one of two SrcS17 mutants (S17A, S17D) was examined. SYF and SrcH cells were transfected with Flag-Ras and SrcWT, SrcS17A, SrcS17D, or pcDNA3 control vector as described in Example 4. Cells were stimulated with isoproterenol or left unstimulated, and cell lysates examined for active GTP-loaded Ras (Flag-Ras-GTP) as described in Example 6.

Unlike SrcWT, the expression of a mutant Src where S17 was replaced with an alanine, (SrcS17A), was unable to reconstitute isoproterenol's activation of Rap 1. Moreover, expression of a second Src mutant in which S17 was replaced with an aspartate (SrcS17D) resulted in constitutive activation of Rap1. This is similar to the results described above in cells treated with Forskolin, an activator of adenylyl cyclases. This activation of Rap1 by SrcS17D was not further stimulated by isoproterenol, indicating that SrcS17D was maximally activating Rap1. The expression of SrcS17A in Src⁺⁺ cells inhibited isoproterenol's activation of Rap 1, indicating that SrcS17A was interfering with signals through endogenous Src in these cells. In contrast to the response in SYF cells, the activation of Rap1 by SrcS 17D and isoproterenol in Src⁺⁺ cells was additive, presumably reflecting the contribution of isoproterenol's activation of endogenous Src.

Conversely, isoproterenol's activation of Ras was not blocked by these mutants. In SYF cells, expression of SrcS 17A reconstituted Ras activation by isoproterenol to a level similar to that observed using SrcWT. In Src⁺⁺ +cells, SrcS17A did not interfere with Ras activation by isoproterenol but enhanced Ras activation. In contrast, SrcS17D was unable to activate Ras in either cell line. These data indicate that SrcS17A is capable of mediating a Src-dependent pathway to Ras, demonstrating that the interference of Rap1 activation by SrcS17A in Src⁺⁺ cells was selective. Moreover, SrcS17D did not potentiate isoproterenol's stimulation of Ras in Src⁺⁺ +cells, indicating that, unlike SrcS17A, SrcS17D alone could not participate efficiently in pathways to activate Ras.

### EXAMPLE 8

### Src's Activation of Ras is Dose Dependent

SrcS17D activated Ras to a moderate degree in SYF cells, but only in conjunction with isoproterenol. This appears inconsistent with SrcS17D's selectivity towards Rap1. One mechanism by which SrcS17D might function as an activator of Rap1 is to bind endogenous proteins that target Src towards Rap1. That is, the requirement of isoproterenol for SrcS17D activation of Ras indicates that SrcS17D is not acting constitutively, but is dependent on additional signals generated downstream of the β-adrenergic receptor. In this model, SrcS17D's selectivity towards Rap1 might not be apparent if it was overexpressed. To examine further the observation that SrcS17D could participate in isoproterenol's activation of Ras in SYF cells, the effect of increasing concentrations of both transfected SrcWT and SrcS 17D in these cells was compared.

SYF cells were transfected with Flag-Ras (5 μg), and 0, 0.5, 1.0, 2.5 or 5.0 μg Flag-SrcWT or Flag-SrcS17D as described above. Cells were left unstimulated or treated with 10 μM isoproterenol, and cell lysates examined for active GTP-loaded Ras (Flag-Ras-GTP) as described in Example 1. Increasing amounts of transfected SrcWT potentiated isoproterenol's activation of Ras at all concentrations examined, especially at low to moderate doses. In contrast, the ability of SrcS17D to carry a signal from isoproterenol to Ras was only apparent at the highest level of expression examined. Therefore, whereas SrcS17D's activation of Rap1 was not further enhanced by isoproterenol, isoproterenol-dependent activation of Ras by SrcS17D was contingent on overexpression. Therefore, SrcS17D interacts with an endogenous protein(s) that channels Src towards a Rap1 pathway, but that when overexpressed, SrcS17D can act independently of this pathway.

### EXAMPLE 9

### PKA Phosphorylation of Src Mediates Rap1 Activation and its Association with B-Raf Following Isoproterenol Stimulation

The mechanism of ERK activation by isoproterenol in Hek293 cells was determined as follows. Hek293 cells were transfected with Flag-Rap1 along with pcDNA3, SrcS17A, SrcS17D, or SrcWT as described above. Cells were treated with 10 μM isoproterenol and cell lysates were examined for active Rap1 using the methods disclosed herein Isoproterenol's ability to activate Rap1 was modestly enhanced following transfection of SrcWT, but was inhibited following transfection of SrcS 17A, indicating that SrcS17A can interfere with the ability of endogenous Src to mediate isoproterenol's activation of Rap1 in Hek293 cells. Expression of SrcS17D constitutively activated Rap1, and this activation was not significantly enhanced by isoproterenol.

Upon its activation, Rap1 binds the effector B-Raf to activate ERKs (Vossler et al., Cell 89:73-82, 1997; Schmitt and Stork, J. Biol. Chem. 275, 25342-50, 2000). This recruitment of B-Raf can be used as an index of Rap1 activation. Hek293 cells were transfected with His-tagged Rap1, in the presence or absence of SrcS 17A, SrcS 17D, CBR, and Cb1-ct plasmids as described herein. Cells were treated with 10 μM isoproterenol, and cell lysates were passed over a nickel column to isolate H is Rap1 and associated proteins, and eluates probed for endogenous B-Raf using Western blotting, as described in Example 3. Isoproterenol's ability to stimulate the association of B-Raf with Rap1 was blocked by SrcS17A. SrcS17D stimulated the association of B-Raf with Rap1, and this was modestly enhanced by isoproterenol. These data demonstrate that phosphorylation of Src S17 is required for both Rap1 activation and function.

As described in the examples above, cAMP activation of Rap1 by Forskolin requires Cb1 and the Rap1 exchanger C3G, in NIH3T3 cells. To examine whether a similar mechanism might underlie signaling from GPCRs, the following methods were used. Hek293 cells were transfected with Flag-tagged SrcWT, SrcS17A, or SrcS17D, using the methods disclosed herein. Cells were treated with 10 μM isoproterenol and equivalent amounts of lysates immunoprecipitated with Flag antibody. The presence of endogenous Cb1 within the eluates was examined by Western blotting. The ability of isoproterenol to induce Rap1-recruitment of B-Raf was blocked by both Cb1-ct and CBR. Isoproterenol also induced the association of Cb1 with SrcWT but not SrcS17A. SrcS17D induced this association in the absence of isoproterenol.

To demonstrate that isoproterenol induces the association of C3G with Cb1 in Hek293 cells, Hek293 cells were transfected with mycCb1 and either SrcS17A or SrcS17D. Cells were treated with isoproterenol or left untreated and equal amounts of lysates immunoprecipitated with Myc antibody and the presence of endogenous C3G within the eluates examined by Westein blot using the methods disclosed herein. Isoproterenol induced an association between C3G and transfected Cb1 that was mimicked by SrcS17D, but not SrcS17A.

To demonstrate that isoproterenol induces the association of Src with C3G in Hek293 cells, Hek293 cells were transfected Flag-tagged SrcWT or SrcS17D and treated with isoproterenol. Equivalent amounts of lysates were immunoprecipitated with Flag antibody and the presence of endogenous C3G within the eluates was examined by Western blots as described herein. An isoproterenol-dependent association between wild type Src and C3G was detected that was also mimicked by SrcS17D. These results indicate that association of Cb1/C3G with Src following isoproterenol stimulation is dependent on phosphorylation of Src S17. In addition, these results identify Cb1 as a target of Src's activation of Rap1.

### EXAMPLE 10

### Constitutively Active Src (SrcY527F) Activates Ras and Rap1

Mutation of tyrosine 527 of Src to phenylanaline (Y527F) produces a constitutively active (oncogenic) Src by eliminating the inhibitory phosphorylation at Y527 (Xu et al. Mol. Cell. 3(5): 629-38, 1999; Brown and Cooper, Biochim. Biophys. Acta 1287(2-3): 121-49, 1996). To determine the effect of this mutant on Ras and Rap1 activation, Hek293 cells were transfected with Flag-tagged Rap1 or Flag-tagged Ras, in the presence or absence of pcDNA3, SrcY527F, SrcS17A/Y527F, SrcS17D/Y527F, CBR, or Cb1-ct using the methods described above. The Src Y527F mutant was synthesized using PCR primers containing sequences corresponding to the 5′ end of the Src cDNA and sequences corresponding to the 3′ end of the Src cDNA, with the sequence corresponding to tyrosine 527 replaced with that for phenylalanine (Y527F). Cell lysates were assayed for Flag-Rap1 activation or Flag-tagged Ras activation, as described above.

The SrcY527F mutant constitutively activated both Ras and Rap1 in Hek293 cells. However, introduction of S17A in SrcY527F created a new mutant (SrcS17A/Y527F) that was unable to activate Rap1, whereas SrcS17D/Y527F activated Rap1 constitutively. Both SrcS17A/Y527F and SrcS17D/Y527F activated Ras, indicating that S17A selectively interfered with oncogenic Src's activation of Rap1. In addition, both Cb1-ct and CBR interfered with Rap1 activation, but had no detectable effect on Ras activation, indicating that the action of endogenous Cb1 and C3G were specific for Rap 1. Therefore, Src-dependent signals can be directed down specific pathways in a stimulus and cell-type specific manner.

### EXAMPLE 11

### Isoproterenol Activation of Rap1 and Ras Occurs

### Through Distinct Heterotrimeric G protein Subunits

The inability of SrcS17A to detectably interfere with Ras signaling was observed by examining hormonally activated Src in Hek293 cells as follows. Hek293 cells were transfected with Flag-Ras in the presence or absence of SrcWT, SrcS17A, SrcS17D, or PARK-ct, and stimulated with isoproterenol using the methods described above. Cell lysates were examined for active Ras. Both SrcWT and SrcS17A, but not SrcS17D, modestly enhanced isoproterenol's activation of Ras. Therefore, Ras activation by isoproterenol is independent of PKA phosphorylation of Src.

To confirm that Ras activation by isoproterenol is mediated via Gβγ, Hek293 cells were transfected with Flag-Ras or Flag-Rap1 in the presence or absence of the transducin (cone) cDNA (Guthrie cDNA Resource Center) and stimulated with either 10 μM isoproterenol or 100 ng/ml EGF as a control. Cell lysates were examined for the activation of Flag-Ras or Flag-Rap1 and for equivalent Flag-Ras or Flag-Rap1 expression within whole cell lysates. Isoproterenol's activation of Ras was blocked by expression of a truncated α-adrenergic receptor kinase (PARK-ct), and by expression of transducin, a retinal-specific Gsα subunit. Both PARK-ct and transducin block signals generated from βγ subunits by binding to endogenous βγ (Birnbaumer, Cell 71:10069-72, 1992; Koch et al., Proc. Natl. Acad. Sci., USA 91:12706-10, 1994). As a control, it was shown that EGF activation of Ras was not blocked by expression of transducin. In contrast, transducin did not block Rap 1 activation by isoproterenol. Taken together, these results demonstrate that while Ras and Rap1 are both activated by Src-dependent mechanisms downstream of the β-adrenergic receptor, only Ras activation involves Gβγ.

In summary, activation of the β-adrenergic receptor triggers two concurrent Src-dependent pathways, involving Gαs to activate Rap1 and Gsβγ to activate Ras, respectively. While Src is required for both Rap1 and Ras activation, only Rap1 activation requires PKA (Example 6). Sequestration of βγ subunits blocked only Ras activation, not Rap1. Therefore, these results indicate that Gsa and PKA are responsible for Rap 1 activation whereas βγ, but not PKA, is responsible for Ras activation.

### EXAMPLE 12

### cAMP Activation of ERKs is PKA- and Src-Dependent

To examine the downstream consequences of Src-dependent signaling in Hek293 cells, ERK activation was measured using pERK antibodies (pERK1/2). Hek293 cells received no pretreatment or were pretreated for 20 minutes with H89 (10 μM), PP2 (10 μM), or LY (10 μM), and stimulated for 5 minutes with isoproterenol (10 μM), Forskolin (10 μM), or 100 ng/ml EGF, using the methods described above. Cell lysates were analyzed by Western blotting for the presence of pERK1/2 (New England Biolabs) or total ERK2 (Santa Cruz Biotechnology) as a control for protein loading.

Isoproterenol's activation of ERKs required both PKA and SFKs, as phosphorylation of ERK was inhibited by both H89 and PP2. Similar results were observed using Forskolin. In contrast, the PI3-K inhibitor, LY, did not block ERK phosphorylation.

### EXAMPLE 13

### Isoproterenol's Activation of ERKs Requires Serine 17 Phosphorylation of Src

To determine the role of PKA phosphorylation of Src serine 17 in ERK activation by cAMP, Hek293 cells were transfected with myc-ERK2 in the presence or absence of SrcS17A or SrcS17D, and stimulated with 10 μM isoproterenol. MycERK2 was immunoprecipitated from cell lysates using an agarose-coupled Myc antibody followed by western blotting for phospho-ERK (pMycERK2, upper panel) or total MycERK2 with a Myc antibody, as a control for protein loading.

Expression of SrcS17A blocked isoproterenol's activation of ERKs, and SrcS17D constitutively activated ERKs, consistent with a model that Src's activation of Rap 1 is necessary and sufficient for isoproterenol's activation of ERKs in these cells. Moreover, isoproterenol was unable to further activate ERKs in SrcS17D-expressing cells, indicating that phosphorylation of SrcS17 is the predominant mode of ERK activation by isoproterenol.

These results support a model whereby the mechanism of activating Src dictates the choice of effector pathways, and identify PKA-phosphorylation of Src as a potential mechanism to direct Src signals towards Rap1.

### EXAMPLE 14

### AKT Activation by Isoproterenol is PKA-independent but Requires Ras and Gβγ

To determine the physiological role of Ras signaling in Hek293 cells, a well-known Ras effector, phosphoinositol-3 kinase (PI3-K) and its target, AKT (a serine/threonine kinase which participates in a variety of cellular effects including cell survival, adhesion, and cell growth) were examined as follows. Hek293 cells received no pretreatment or were pretreated for 20 minutes with H89 (10 μM), PP2 (10 μM), or LY (10 μM), and stimulated for 5 minutes with isoproterenol. Cell lysates were analyzed by Western blotting for phospho-AKT (PAKT 308, Cell Signaling, Beverly, Mass.) or total AKT (Cell Signaling) as a control. Isoproterenol activated AKT, as measured by phosphorylation-specific antibodies to phosphothreonine 308 (AKT). This phosphorylation required P13-K as LY blocked isoproterenol-induced phosphorylation at this site. Moreover, this phosphorylation was blocked by PP2, but not H89, indicating the requirement of SFKs, but not PKA. Therefore, isoproterenol's activation of AKT proceeds through Src and PI3-K but not PKA.

The requirement for Ras in isoproterenol's activation of AKT in Hek293 cells was demonstrated by the ability of the interfering mutant of Ras, RasN17, to block this effect Hek293 cells were transfected with HA-AKT (Hemagglutinin-tagged AKT, Dr. Thomas Soderling, Vollum Institute, Portland, Oreg.) in the presence or absence of SrcWT, SrcS17A, SrcS17D, RasN17, or PARK-ct, and stimulated with isoproterenol, using the methods described above. Similar amounts of cell lysate (determined by protein concentrations) were immunoprecipitated using an agarose-coupled HA antibody followed by Western blotting for phospho-AKT (PHA-AKT 308) or total HA-AKT with an HA antibody (12CA5, Boehringer Mannheim, Indianapolis, Ind.). SrcS17A did not block AKT's activation by isoproterenol nor did SrcS17D result in constitutive activation of AKT. Like their actions on Ras, SrcS17A and SrcWT enhanced phosphorylation of AKT to similar levels, indicating that, although overexpression of SrcS17A selectively interfered with effectors of Rap 1, it mimicked the action of wild type Src on effectors downstream of Ras.

In summary, β-adrenergic receptor activation of Ras required Ste, but unlike Rap1, Ras activation required Gsβγ. This activation of Ras is capable of coupling positively to AKT. Therefore, in Hek293 cells, the β-adrenergic receptor is capable of activating AKT through a pathway involving βγ, Src, Ras, and PI3-K.

### EXAMPLE 15

### Model of cAMP Inhibition of ERKs and Cell Growth

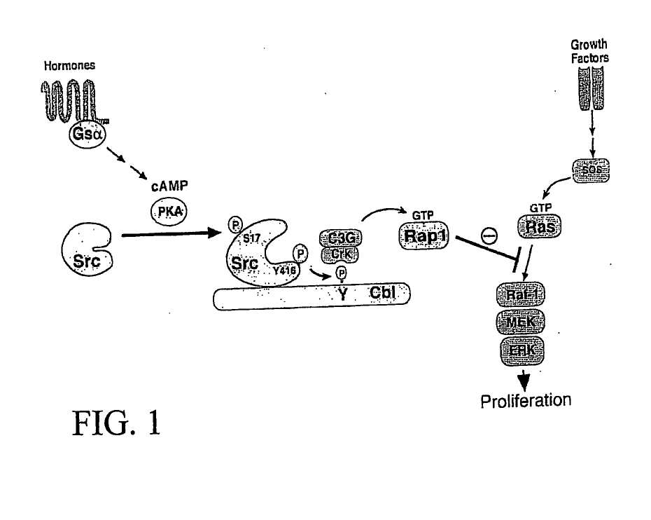

Without wishing to be bound to a particular theory, a model is proposed that describes the role of Src activation, specifically phosphorylation of Ser17, in cell differentiation (FIG. 1). Hormones binding to heptahelical GPCRs activate Gsa and adenylyl cyclase to elevate intracellular levels of cAMP. Upon cAMP activation of PKA, PKA phosphorylates Src at Ser17, triggering Src activation. This can be monitored by the autophosphorylation of tyrosine 416. This Src activation results in the phosphorylation of downstream Src effectors, including Cb1. Cb1 phosphorylation of tyrosines 700 and 774 by Src induces the binding of the Crk/C3G complex via the SH2 domains of Crk by bringing C3G to the membrane, where C3G activates Rap 1. Upon activation, Rap1 binds to Raf-1, sequestering it from Ras, and preventing Ras activation of Raf-1, MEK, and ERK. Growth factors can potently activate Ras (by recruiting the Ras GEF, SOS, to the membrane). However, when Rap1 is activated, Rap1 prevents Ras from signaling to Raf-1, thereby inhibiting signals from Ras to ERKs and cell proliferation.

By identifying an anti-proliferative action of Src coupled to the second messenger cAMP, the possible physiological roles of this proto-oncogene are extended. In addition, although inhibition of SFKs blocked cAMP's inhibition of ERK, it did not block the activation of ERKs by either PDGF or EGF. Hence, Src activation of Rap1 and inhibition of ERKs are believed to be more significant than Src's potentiation of ERK signaling. In other cell types, Src may exert anti-proliferative effects downstream of cAMP, as well as proliferative effects downstream of growth factors within the same cell.

It was also observed that specific modes of activation of Src can trigger distinct effector pathways of Src. For example, SrcS17A selectively inhibits cAMP-dependent activation of Src, while not effecting growth factor-dependent activation of Src. Therefore, hormonal activation of Src via PKA may utilize a mechanism of activation distinct from that used in growth factor activation of Src. Selective response of Src following activation by PKA may reflect the restricted activation of downstream effectors. This restricted response may be due to phosphorylation-dependent changes in subcellular localization of Src and/or the regulation of the binding of accessory proteins to Src's amino-terminus. Like other known binding proteins, this binding regulates the activity of the Src kinase domain.

The mechanism that permits SrcS17D to direct Src kinase activity towards selective substrates may be that the proximity of S17 site to the N-terminal myristoylation of Src may allow this phosphorylation (or aspartate residue) to influence proper membrane targeting. The ability of Src mutants to interfere with Rap1 activation indicates that the cellular interactions that are disrupted by this mutant are saturatable. In addition, that SrcS17D is constitutively active suggest that it is not just being relocalized, but is activated as well. It is also possible that this phosphorylation may influence the binding of additional docking/adaptor proteins that influence both Src's activity and choice of substrates.

In summary, the role of Src in cAMP's activation of Rap1, inhibition of ERKs, and inhibition of cell growth has been demonstrated. These actions require the direct phosphorylation of Src on Ser17 by PKA itself. This represents a novel example of a physiological regulation of Src function by PKA. In addition, it identifies for the first time a physiological role of Src in the anti-proliferative actions of hormones linked to increased levels of intracellular cAMP.

In addition, in Hek293 cells, isoproterenol activates Rap1, stimulates Rap1 association with B-Raf, and activates ERKs, all via PKA. In contrast, isoproterenol's activation of Ras requires Gβγ subunits, is independent of PKA, and results in the phosphoinositol-3 kinase (PI3-K)-dependent activation of AKT. β-adrenergic stimulation of both Rap1 and ERKs, but not Ras and AKT, can be blocked by the Src mutant SrcS17A, that is incapable of being phosphorylated and activated by PKA. Furthermore, the Src mutant SrcS17D, which mimics PKA phosphorylation at serine 17, stimulates Rap1 activation, Rap1/B-Raf association, and ERK activation but does not stimulate Ras or AKT. These results indicate that Rap1 activation, but not Ras activation, is mediated through the direct phosphorylation of Src by PKA. Therefore, the β₂-adrenergic receptor activates Src via two independent mechanisms to mediate distinct signaling pathways, one through Gsa to Rap1 and ERKs and the other through Gβγ to Ras and AKT.

### EXAMPLE 16

### Production of Antibodies

As described above in EXAMPLE 4, antibodies are available which recognize Src when phosphorylated at Ser17. However, the antibody disclosed in EXAMPLE 4 recognizes phospho-serine/threonine residues in any protein, not only Src. Therefore, it would be advantageous to have an antibody that specifically recognized Src Ser17 when it was phosphorylated, that is, an antibody that was specific for Src Ser17 phosphorylation, not merely a phospho-PKA substrate antibody.

Antibodies which specifically recognize Src when phosphorylated at Ser17 are beneficial for diagnosis and prognosis of Src positive tumors. For example, the presence of a detectable positive binding reaction between such phospho-specific antibodies and a tumor sample indicates that Src is phosphorylated at Ser17. This result indicates that the tumor is no longer proliferating, and/or that the subject is responding to an anti-neoplastic therapy, and/or that the anti-neoplastic therapy can be terminated. In contrast, the absence of a detectable positive binding reaction between the phospho-specific antibodies and a tumor sample indicates that Src is not phosphorylated at Ser17. This result indicates that the tumor is actively proliferating, and/or that the subject requires further anti-neoplastic therapy, and/or an alternative anti-neoplastic therapy.

In addition, phosphospecific antibodies can be used to further investigate the role of phosphorylation of Src at Ser17, such as its effect on proliferation.

Methods of making phosphospecific antibodies are well known in the art (for example see U.S. Pat. No. 5,814,459 to Montminy and U.S. Pat. No. 6,309,863 to Anderson et al). Phosphospecific antibodies that are specifically immunoreactive with Src when it is phosphorylated at Ser17 of can be generated using Src proteins, fragments, mutants, analogs, variants, fusions, or derivatives, that include the phosphorylated Ser17, as immunogens. The resulting antibodies can be for example, polyclonal, monoclonal, chimeric, humanized, single chain, Fab fragments, or from an Fab expression library.

In one example, the phosphospecific antibodies are reactive with Src polypeptide fragments including about 8 to about 20 amino acids, wherein about 4 to about 10 amino acids are positioned on each side of the Ser17 phosphorylation site. In another example, the phosphospecific antibodies are reactive with Src polypeptide fragments including about 10 to about 20 amino acids, wherein about 5 to about 10 amino acids are positioned on each side of the serine-17 phosphorylation site. Examples of Src polypeptide fragments that may be recognized by the phosphospecific antibodies, and/or can be used to generate the antibodies, include but are not limited to: QRRRSLEPA (SEQ ID NO: 5); SQRRRSLEPAE (SEQ ID NO: 6) ASQRRRSLEPAEN (SEQ ID NO: 7); DASQRRRMLEPAENV (SEQ ID NO: 8); KDASQRRRSLEPAENVH (SEQ ID NO: 9); PKDASQRRRSLEPAENVHG (SEQ ID NO: 10); KPSKPKDASQRRRSLEPAENVHGA (SEQ ID NO: 11) (wherein Ser17 (underlined) is phosphorylated).

One skilled in the art will understand that variants of these peptides could also be used, for example by substituting one or more amino acids with a conservative substitution, as long as the antibody retains the ability to specifically bind to Src when it is phosphorylated at Ser17. Furthermore, longer or shorter sequences can be used to generate an antibody, for example by deleting and/or adding one or more of the amino acids from and/or to the N- or C-terminus of the peptides listed above, as long as the antibody retains the ability to specifically bind to Src when it is phosphorylated at Ser17. In addition, Src proteins and peptides can be modified using a variety of chemical techniques to produce derivatives having essentially the same activity as the unmodified peptides, and optionally having other desirable properties, such as higher antigenicity.

One method that can be used to produce such antibodies is to recombinantly express a region including phosphorylated Ser17 of Src, for example in bacteria. Alternatively, the peptides based upon amino acid sequences of a Src protein (or variant, fusion, or fragment thereof) can be chemically synthesized, for example as described in EXAMPLE 22. The resulting protein or protein fragment is used to generate antibodies that specifically recognize Src when it is phosphorylated at Ser17. To assist in purification, β-globin, 6-Histidine, or glutathione S-transferase (GST), can be conjugated to the peptide, forming a fusion protein. Substantially pure Src protein phosphorylated at Ser17, or peptides thereof including phosphorylated Ser17, is suitable for use as an immunogen may be isolated from the transfected or transformed cells. For example, Src protein or peptide that is phosphorylated at Ser17 which is at least 50% pure, for example at least 75% pure, can be used. Concentration of protein in the final preparation is adjusted, for example, by concentration on an Amicon filter device, to the level of a few micrograms per milliliter. Antibodies are then prepared as described below.

Testing Antibody Specificity

Ideally, antibodies raised against Src when it is phosphorylated at Ser17 specifically detect the Src protein when it is phosphorylated at Ser17. That is, antibodies raised against the protein recognize and bind the Src protein when it is phosphorylated at Ser17 and do not substantially recognize or bind to other proteins found in human cells, including other phosphorylated proteins, nor would it bind to Src when it is unphosphorylated at Ser17. The determination that an antibody specifically detects a Src protein when it is phosphorylated at Ser17 is made by any one of a number of standard immunoassay methods; for instance, the Western blotting technique (Sambrook et al., 1989). To determine that a given antibody preparation specifically detects the Src protein when it is phosphorylated at Ser17 by Western blotting, total cellular protein is extracted from a sample (for example, a sample prepared from breast cancer cells that over-expresses Src) and electrophoresed on a sodium dodecyl sulfate-polyacrylamide gel. The proteins are then transferred to a membrane (for example, nitrocellulose) by Western blotting, and the antibody preparation is incubated with the membrane.

After washing the membrane to remove non-specifically bound antibodies, the presence of specifically bound antibodies is detected by the use of an anti-mouse or anti-rabbit antibody conjugated to an enzyme such as alkaline phosphatase; application of the substrate 5-bromo-4-chloro-3-indolyl phosphate/nitro blue tetrazolium results in the production of a dense blue compound by immuno-localized alkaline phosphatase. Antibodies that specifically detect a Src protein when it is phosphorylated at Ser17 will bind to a Src protein when it is phosphorylated at Ser17 (which will be localized at a given position on the gel determined by its molecular weight or phosphorylated state). Non-specific binding of the antibody to other proteins may occur and may be detectable as a weak signal on the Western blot The non-specific nature of this binding will be recognized by one skilled in the art by the weak signal obtained on the Western blot relative to the strong primary signal arising from the specific antibody-Src phosphorylated at Ser17 binding.

An alternative or additional method that can be used to test the specificity of the phosphospecific antibody is immunoprecipitation. Briefly, cell lysates that contain Src phosphorylated at Ser17 are incubated with the antibody preparation for a few hours, captured on protein-A beads with an overnight incubation, washed several times and analyzed by SDS-PAGE followed by Western blotting and detection with the antibody as described above. Thus, if the antibodies recognize Src when it is phosphorylated at Ser17, the antibodies should immunoprecipitate Src and produce a signal in Western blotting.

Monoclonal Antibody Production by Hybridoma Fusion

Monoclonal antibody to a Src protein phosphorylated at Ser17 can be identified, isolated and prepared from murine hybridomas using the method of Kohler and Milstein (Nature 256:495, 1975) or derivative thereof. Briefly, a mouse is repetitively inoculated with a few μg of the selected peptide that includes the phosphorylated Ser17 of Src, over a period of a few weeks. If desired, the immunogenic peptide can be chemically modified, for example modification of the N- or C-terminus with N-succinimidyl, S-acetyl-thioacetate (SATA), and/or keyhole limpet hemocyanin (KLH, a common carrier for antigens).

The mouse is sacrificed and antibody-producing cells of the spleen isolated. The spleen cells are fused using polyethylene glycol with mouse myeloma cells, and the excess unfused cells destroyed by growth of the system on selective media comprising aminopterin (HAT media). The fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as described by Engvall (Enzymol. 70:419, 1980), and similar methods. Selected positive clones can be expanded and their monoclonal antibody product harvested for use.

Detailed procedures for monoclonal antibody production are described in Harlow and Lane (Antibodies: A Laboratory Manual. 1988, Cold Spring Harbor Laboratory, New York). In addition, protocols for producing humanized forms of monoclonal antibodies (for therapeutic applications) and fragments of monoclonal antibodies are known in the art

Polyclonal Antibody Production by Immunization

Polyclonal antiserum containing antibodies to heterogeneous epitopes of Src that include the phosphorylated Ser17, can be prepared by immunizing suitable animals with the desired protein (see EXAMPLES 20 and 22), which can be unmodified or modified to enhance immunogenicity. Effective polyclonal antibody production is affected by many factors related both to the antigen and the host species. For example, small molecules (for example SEQ ID NOS: 5-11) may be less immunogenic than others and may require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis et al. (J. Clin. Endocrinol. Metab. 33:988-91, 1971).

Booster injections can be given at regular intervals, such as monthly, and antiserum harvested when antibody titer thereof, as determined semi-quantitatively, for example, by double immunodiffusion in agar against known concentrations of the antigen, begins to fall. See, for example, Ouchterlony et al. (In: Handbook of Experimental Immunology, Wier, D. (ed.). Chapter 19. Blackwell. 1973). Plateau concentration of antibody is usually in the range of 0.1 to 0.2 mg/ml of serum (about 12 μM). Affinity of the antisera for the antigen is determined by preparing competitive binding curves, as described, for example, by Fisher (Manual of Clinical Immunology, Chapter 42. 1980).

Antibodies Raised by Injection of a Src cDNA

Antibodies can be raised against a Src protein phosphorylated at Ser17, by subcutaneous injection of a DNA vector expressing a Src protein or peptide wherein Ser17 is phosphorylated, into an animal, such as mice or rabbits. Delivery of the recombinant vector into the animal can be achieved using a hand-held form of the Biolistic system (Sanford et al., Particulate Sci. Technol. 5:27-37, 1987) described by Tang et al. (Nature 356:1524, 1992). Expression vectors include recombinant vectors expressing Src cDNA under transcriptional control of the human β-actin promoter or cytomegalovirus (CMV) promoter.

Antibody preparations prepared according to these protocols are useful in quantitative immunoassays to determine concentrations of antigen-bearing substances in samples; or semi-quantitatively or qualitatively to identify the presence of antigen in a sample.

Labeled Antibodies

The Src antibodies disclosed herein can be conjugated with a label for their direct detection (see Chapter 9, Harlow and Lane, Antibodies: A Laboratory Manual. 1988). The label, which can include, but is not limited to, a radiolabel, an enzyme (i.e. alkaline phosphatase (AP) or horseradish peroxidase (HRP)), a fluorophore, or biotin, is chosen based on the method of detection available to the user. The method of producing these conjugates is determined by the reactive group on the label added.

For example, antibodies disclosed herein can be radiolabeled with iodine (¹²⁵I), which yields low-energy gamma and X-ray radiation. Briefly, 10 μg of protein in 25 μl of 0.5 M sodium phosphate (pH 7.50 is placed in a 1.5 ml conical tube. To this, 500 μC of Na¹²⁵I, and 25 μl of 2 mg/ml chloramine T is added and incubated for 60 seconds at RT. To stop the reaction, 50 μL of chloramine T stop buffer is added (2.4 mg/ml sodium metabisulfite, 10 mg/ml tyrosine, 10% glycerol, 0.1% xylene cyanol in PBS). The iodinated antibody is separated from the iodotyrosine on a gel filtration column.

### EXAMPLE 17

### Detection and Quantitation of Src

This example teaches how Src can be detected in a sample, and further, how to determine whether Src is or is not phosphorylated at Ser17, or has a relative increase or decrease in Ser17 phosphorylation. In one example, the method is used to determine if a Src-expressing tumor cell express Src which is, or is not, phosphorylated at Ser17. In another example, the method is used to quantitate the amount of Src phosphorylated at Ser17 and/or the amount of Src unphosphorylated at Ser17, in the sample, so that relative phosphorylation states can be measured to observe changes in phosphorylation.

For determining whether a sample contains Src which is, or is not, phosphorylated at Ser17, and/or for quantitating the protein, a sample which includes cellular proteins is used. For example, a sample can be prepared from or obtained from a tumor cell. Several types of tumors overexpress Src, such as tumors of the breast, colon, pancreas, brain, lung, ovary, esophagus, and gastric tract, as well as melanoma, adenocarcinomas, retinoblastomas, neuroblastomas, and leukemias (Irby and Yeatman. Oncogene 19:563642, 2000). These tumors overexpress Src, relative to Src expression in the same tissue type that is not neoplastic.

Furthermore, the level of Src activity increases with the stage of the disease. As described above (for example see EXAMPLES 4 and 5), the phosphorylation state of Src at Ser17 plays a role in proliferation. Specifically, phosphorylation of Src at Ser17 has anti-proliferative effects. Therefore, it is useful to determine whether a Src-positive tumor is expressing the phosphorylated Ser17 form of Src, or the unphosphorylated Ser17 form. Such information is used for diagnosis, prognosis, and/or to design therapy. Quantitation of Src protein in a sample by immunoassay is performed and then compared to levels of the protein found in non-Src expressing cells. In addition, quantitation of the amount of Src phosphorylated or not at Ser17 by immunoassay is performed and then compared to levels of the protein found in other Src-expressing cells, such as control cells.

The antibodies described in EXAMPLE 16 can be used in diagnostic applications to detect and quantitate Src expression in tumors and pre-cancerous lesions, specifically, to determine whether the tumor or lesion is expressing Src phosphorylated at Ser17, or expressing Src unphosphorylated at Ser17. In addition, such antibodies can be used to quantitate the level or amount of Src phosphorylated at Ser17 expression in a sample and/or the amount of Src unphosphorylated at Ser17 expression in a sample, such as a tumor cell. If the results demonstrate that the tumor is expressing the unphosphorylated Ser17 form of Src, this indicates that the tumor is in a proliferative state. This may assist a physician in choosing a therapy, such as a therapy that phosphorylates Src at Ser17, which may have anti-proliferative effects.

In one example, the antibodies are used to monitor the change in Src expression during the course of anti-neoplastic therapy. If the subject is undergoing an anti-neoplastic therapy, demonstration that the tumor is expressing the unphosphorylated Ser17 form of Src (or is expressing more of the unphosphorylated Ser17 form of Src than the phosphorylated Ser17 form of Src), indicates that the therapy is not effective, or is less effective. In contrast, demonstration that the tumor is expressing the phosphorylated Ser17 form of Src (or is expressing more of the phosphorylated Ser17 form of Src than the unphosphorylated Ser17 form of Src), indicates that the therapy is effective, or is more effective than treatments in which the unphosphorylated Ser17 form of Src is detected.

General Immunoassays

Antibodies that can distinguish between Src when it is or is not phosphorylated at Ser17 facilitate quantitation of these forms of Src protein using any immunoassay method known in the art (Harlow and Lane, Antibodies: A Laboratory Manzual. 1988). Such assays permit one to determine if Src is present in a sample, and if Src is or is not phosphorylated at Ser17, and quantitation of such proteins. Typical methods involve combining the sample with a Src-specific binding agent, such as an antibody, such as the antibodies disclosed in EXAMPLE 16, so that complexes form between the binding agent and the Src protein present in the sample, and then detecting or quantitating such complexes.

These assays can be performed with a specific binding agent immobilized on a support surface, such as in the wells of a microtiter plate or on a column. A sample is then introduced onto the support surface and allowed to interact with the specific binding agent so as to form complexes. Excess sample is removed by washing, and the complexes are detected with a reagent, such as a second anti-Src antibody conjugated with a detectable marker.

In another example, the cellular proteins are isolated and subjected to SDS-PAGE followed by Western blotting. After resolving the proteins, the proteins are transferred to a membrane, which is probed with specific binding agents that recognize Src when it is phosphorylated at Ser17. The proteins are detected, for example with HRP-conjugated secondary antibodies, and quantitated.

Flow Cytometry

Flow cytometric analysis can be used to analyze intact live cells, obtained from blood, aspriates, bone marrow, or other source. Cells are labeled by methods known to those skilled in the art with a fluorescently-conjugated Src antibody, for example an antibody that can distinguish between Src when it is and is not phosphorylated at Ser17. The cells can be fixed and/or permeabilized to allow the antibody access to the intracellular Src. Cells are washed to remove unbound antibody, then resuspended in an appropriate flow cytometry buffer, such as PBS. Flow cytometric analysis will allow one to determine the percent of cells expressing Src, and in particular examples, the percent of cells expressing Src phosphorylated or not at Ser17.

Imaging Methods

For paraffin embedded preparations, the sample is formalin fixed, paraffin embedded and cut at 10 μm. Slides containing the tissue sections are incubated in 0.05% pronase (Boehringer Mannheim, 7000U/g), preheated in 1.47 mM CaCl₂ to 37° C. for 10 minutes. For frozen biopsied tissue, the frozen sections are thawed at RT and fixed with acetone at −200° C. for five minutes. Slides are washed twice in cold PBS for five minutes each, then air-dried.

Sections are covered with or without (negative control) 20-30 μl of Ab solution (15-45 μg/l) (diluted in PBS, 2% BSA at 15-50 μg/ml) and incubated at RT in humidified chamber for 30 minutes. Slides are washed three times with cold PBS five minutes each, allowed to air-dry briefly (five minutes) before applying 20-30 μl of the second antibody solution (diluted in PBS, 2% BSA at 15-50 μg/ml) and incubated at RT in humidified chamber for 30 minutes. The label on the second antibody may contain a label, such as a fluorescent probe, enzyme, radiolabel, biotin, or other detectable marker. The slides are washed three times with cold PBS five minutes each then quickly dipped in distilled water, air-dried, and mounted with PBS containing 30% glycerol. Nuclei can be counterstained with hernatoxylin. Images of the immunostained tissue can then be obtained using a light microscope. Slides can be stored at 4° C. prior to viewing.

For samples prepared for electron microscopy (versus light microscopy), the second antibody is conjugated to gold particles. Tissue is fixed and embedded with epoxy plastics, then cut into very thin sections (˜1-2 μm). The specimen is then applied to a metal grid, which is then incubated in the primary anti-Src antibody, washed in a buffer containing BSA, then incubated in a secondary antibody conjugated to gold particles (usually 5-20 nm). These gold particles are visualized using electron microscopy methods.

### EXAMPLE 18

### Methods of Screening Test Agents

Methods of screening test agents to identify biologically active molecules that increase or decrease proliferation are disclosed. Screening of potential agents that increase or decrease proliferation is facilitated by the knowledge that proliferation can be regulated by the phosphorylation state of Src Ser17. For example, agents can be screened for their ability to specifically phosphorylate or de-phosphorylate (or decrease or even inhibit phosphorylation) Src at Ser17. In addition, agents can be screened for an ability to mimic the biological activity of Src when it is, or is not, phosphorylated at Ser17.

The disclosure therefore includes synthetic embodiments of naturally-occurring peptides described herein, as well as mimetics (non-peptide organic molecules), derivatives (chemically functionalized peptide molecules obtained starting with a Src peptide sequence) and variants (homologs) of these peptides that specifically increase or decrease proliferation. Each peptide of the disclosure includes a sequence of amino acids, which may be either L- and/or D-amino acids, naturally occurring and otherwise. Compounds or other molecules that mimic Src activity can be identified and/or designed.

Mimetics include molecules, such as an organic chemical compound, that mimic the activity of Src. For example, a molecule that mimics the activity of Src when it is phosphorylated at Ser17, has anti-proliferative activity. In contrast, a molecule that mimics the activity of Src when it is not phosphorylated at Ser17, has proliferative activity.

Peptidomimetic and organomimetic embodiments are within the scope of this term, wherein the three-dimensional arrangement of the chemical constituents of such peptido- and organomimetics mimic the three-dimensional arrangement of the peptide backbone and component amino acid sidechains in the peptide, resulting in such peptido- and organomimetics of the peptides having substantial specific proliferative or anti-proliferative activity. For computer modeling applications, a pharmacophore is an idealized, three-dimensional definition of the structural requirements for biological activity. Peptido- and organomimetics can be designed to fit each pharmacophore with current computer modeling software (using computer assisted drug design). See Walters, Computer-Assisted Modeling of Drugs, in Klegerman & Groves, eds., 1993, Pharmaceutical Biotechnology, Interpharm Press: Buffalo Grove, Ill., pp. 165-174 and Principles of Pharmacology (ed. Munson, 1995), chapter 102 for a description of techniques used in computer assisted drug design.

Synthetic drug databases (which can be licensed from several different drug companies) can be screened to identify agents that can substitute for Src. For example, test agents can be incubated with the SYF cells disclosed in EXAMPLE 3 (these cells do not express Src, Yes, and Fyn) and the effect on proliferation measured using the Mu assay described in EXAMPLE 3. The effect on proliferation can be compared to SYF cell proliferation in the absence of the test agent Moreover, structure activity relationships and computer assisted drug design can be performed as described in Remington, The Science and Practice of Pharmacy, 1995, Chapter 28.

Synthetic peptides can be designed from the Src sequence or mutants thereof (such as the S17A and S17D mutants). Several different peptides can be generated from this region. Chimeric peptides can be expressed recombinantly as described herein, for example in SYF cells, and their effect on proliferation, Ras activation, and/or Rap1 activation, determined. One advantage of synthetic peptides over the monoclonal antibodies is that they are smaller, and therefore diffuse easier, and are not as likely to be immunogenic. Standard mutagenesis of such peptides can also be performed to identify variant peptides having even greater Src biological activity, such as those that have a greater ability to increase or decrease proliferation.

As an alternative to using the SYF cells, labeled Src protein (such as a radioactively labeled protein) can be used to probe compounds or peptides arranged in an array on a substrate. The array can be used as a probe to determine which of the compounds or peptides hybridize to the labeled protein, such as a comparison of compounds that only bind to Src when it is or is not phosphorylated at Ser17. Such hybridized compounds or peptides can then be tested as described herein, for example using the MTT proliferation assay, a Rap1 activation affinity assay, a Ras activation affinity assay, and in a mouse model (see EXAMPLE 27) for biological function. These results can be compared to the effect on proliferation, Ras or Rap1 activation, in the absence of the test agent.

Test agents such as synthetic drugs or peptides that are observed to increase or decrease proliferation, are good candidates for therapies, such as treatment of diseases including, but not limited to cancer, such as Src-positive tumors, heart disease, Alzheimer's, Parkinson's disease, and osteoporosis.

### EXAMPLE 19

### Production of Sequence Variants

Disclosed herein are specific binding agents, such as antibodies, that specifically recognize Src when it is phosphorylated at Ser17, and methods for screening for compounds that increase or decrease proliferation using Src or a fragment thereof that includes phosphorylated Ser17. It is understood by those skilled in the art that use of non-native Src sequences (such as polymorphisms, fragments, mutants, or variants) can be used to produce phosphospecific antibodies and to practice the screening methods of the present disclosure, as long as the distinctive functional characteristics of the Src antigen are retained. For example, Src variants can be used to practice the methods disclosed herein if when phosphorylated at Ser17, they retain their anti-proliferative ability. This activity can readily be determined using the assays disclosed herein, for example the MTT assay described in the above Examples. In yet other examples, Src variants can be used to practice the methods disclosed herein if when not phosphorylated at Ser17, they retain their proliferative ability.

This disclosure facilitates the use of DNA molecules, and thereby proteins, derived from a native protein but which vary in their precise nucleotide or amino acid sequence from the native sequence. Such variants can be obtained through standard molecular biology laboratory techniques and the sequence information disclosed herein DNA molecules and nucleotide sequences derived from a native DNA molecule can also be defined as DNA sequences that hybridize under stringent conditions to the DNA sequences disclosed, or fragments thereof. Hybridization conditions resulting in particular degrees of stringency vary depending upon the nature of the hybridization method and the composition and length of the hybridizing DNA used. Generally, the temperature of hybridization and the ionic strength (especially the Na⁺ concentration) of the hybridization buffer determines hybridization stringency. Calculations regarding hybridization conditions required for attaining particular amounts of stringency are discussed by Sambrook et al. (Molecular Cloning: A Laboratory Manual, Cold Spring Harbor, New York, 1989, Chapters 9 and 11). Hybridization with a target probe labeled with [³²P]dCT? is generally carried out in a solution of high ionic strength such as 6×SSC at a temperature that is about 5-25° C. below the melting temperature, Tₘ. The term Tₘ is the temperature (under defined ionic strength and pH) at which 50% of the target sequence remains hybridized to a perfectly matched probe or complementary strand. The Tₘ of such a hybrid molecule may be estimated from the following equation (Bolton and McCarthy, Proc. Natl. Acad. Sci. USA 48:1390, 1962): Tₘ=81.5° C.-16.6(log₁₀[Na⁺])+0.41(% G+C)-0.63(% form amide)-(600/1); where 1=the length of the hybrid in base pairs.

An example of stringent conditions is a salt concentration of at least about 0.01 to 1.0 M Na ion concentration (or other salts) at pH 7.0 to 8.3 and a temperature of at least about 30° C. for short probes (e.g. 10 to 50 nucleotides). Stringent conditions can also be achieved with the addition of destabilizing agents such as formamide. For example, conditions of 5×SSPE (750 mM NaCl, 50 mM Na phosphate, 5 mM EDTA, pH 7.4) at 25-30° C. are suitable for allele-specific probe hybridizations.

The degeneracy of the genetic code further widens the scope of the present disclosure as it enables major variations in the nucleotide sequence of a Src DNA molecule while maintaining the amino acid sequence of the encoded Src protein. For example, the amino acid Ala is encoded by the nucleotide codon triplet GCT, GCG, GCC and GCA. Thus, the nucleotide sequence could be changed without affecting the amino acid composition of the encoded protein or the characteristics of the protein. Based upon the degeneracy of the genetic code, variant DNA molecules may be derived from a cDNA molecule using standard DNA mutagenesis techniques as described above, or by synthesis of DNA sequences. DNA sequences that do not hybridize under stringent conditions to the cDNA sequences disclosed by virtue of sequence variation based on the degeneracy of the genetic code are also comprehended by this disclosure.

Amino acid substitutions are typically of single residues; for example 1, 2, 3, 4, 5, 10 or more substitutions; insertions usually will be on the order of about from 1 to 10 amino acid residues; and deletions will range about from 1 to 30 residues. Substitutions, deletions, insertions or any combination thereof may be combined to arrive at a final construct Ideally, mutations in the DNA encoding the protein should not place the sequence out of reading frame and will not create complementary regions that could produce secondary mRNA structure.

The simplest modifications involve the substitution of one or more amino acid residues (for example 2, 5 or 10 residues) for amino acid residues having similar biochemical properties. These so-called conservative substitutions are likely to have minimal impact on the activity of the resultant protein. Substitutional variants are those in which at least one residue in the amino acid sequence has been removed and a different residue inserted in its place. Such substitutions generally are conservative when it is desired to finely modulate the characteristics of the protein. Substantial changes in function or immunological identity are made by selecting substitutions that are less conservative.

Such variants can be readily selected for additional testing by performing an assay (such as those described in EXAMPLES 1-14) to determine if the variant retains anti-proliferative activity when phosphorylated at Ser17.

### EXAMPLE 20

### Recombinant Expression

With publicly available Src cDNA and amino acid sequences (for example see Genbank Accession Nos. P12931 and TVHUSC as well as SEQ ID NOS: 1 and 2), as well as the disclosure herein of variants, polymorphisms, mutants, fragments and fusions thereof, the expression and purification of any Src protein by standard laboratory techniques is enabled. One skilled in the art will understand that recombinant methods can be used to express Src that is, or is not, phosphorylated at Ser17 (e.g. WT Src, SrcS17A, or SrcS17D). Such expression can be performed in any cell or organism of interest, and the protein purified prior to use, for example prior to immunization of a subject to produce antibodies, or prior to screening test compounds.

Methods for producing recombinant proteins are well known in the art Therefore, the scope of this disclosure includes recombinant expression of any Src protein, fragment, fusion, or variant thereof. For example, see U.S. Pat. No. 5,342,764 to Johnson et al.; U.S. Pat. No. 5,846,819 to Pausch et al.; U.S. Pat. No. 5,876,969 to Fleer et al. and Sambrook et al. (Molecular Cloning: A Laboratory Manual, Cold Spring Harbor, N.Y., 1989, Ch. 17, herein incorporated by reference).

In addition, methods for producing recombinant Src are known, for example as disclosed in U.S. Pat. No. 6,100,254 to Budde et al. and U.S. Pat. No. 6,051,593 to Tang et al. (and references therein), Marx et al., Exp. Mol. Pathol. 70:201-13, 2001, and Trouet et al., Am. J. Physiol. Cell. Physiol. 281: C248-56, 2001.

Briefly, partial, full-length, fusion, or variant Src cDNA sequences, which encode for a Src protein or peptide, can be ligated into an expression vector, such as a bacterial expression vector. Proteins and/or peptides can be produced by placing a promoter upstream of the cDNA sequence. Examples of promoters include, but are not limited to lac, trp, tac, trc, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

Vectors suitable for the production of intact native proteins include pKC30 (Shimatake and Rosenberg, 1981, Nature 292:128), pKK177-3 (Amann and Brosius, 1985, Gene 40:183) and pET-3 (Studiar and Moffatt, 1986, J. Mol. Biol. 189:113). A DNA sequence can be transferred to other cloning vehicles, such as other plasmids, bacteriophages, cosmids, animal viruses and yeast artificial chromosomes (YACs) (Burke et al., 1987, Science 236:806-12). These vectors can be introduced into a variety of hosts including somatic cells, and simple or complex organisms, such as bacteria, fungi (Timberlake and Marshall, 1989, Science 244:1313-7), invertebrates, plants (Gasser and Fraley, 1989, Science 244:1293), and mammals (Pursel et al., 1989, Science 244:1281-8), which are rendered transgenic by the introduction of the heterologous Src cDNA.

For expression in mammalian cells, a Src cDNA sequence can be ligated to heterologous promoters, such as the simian virus SV40, promoter in the pSV2 vector (Mulligan and Berg, 1981, Proc. Natl. Acad. Sci. USA 78:2072-6), and introduced into cells, such as monkey COS-1 cells (Gluzmnan, 1981, Cell 23:175-82), to achieve transient or long-term expression. The stable integration of the chimeric gene construct may be maintained in mammalian cells by biochemical selection, such as neomycin (Southern and Berg, 1982, J. Mol. Appl. Genet. 1:32741) and mycophoenolic acid (Mulligan and Berg, 1981, Proc. Nad. Acad. Sci. USA 78:2072-6).

The transfer of DNA into eukaryotic, such as human or other mammalian cell, is a conventional technique. The vectors are introduced into the recipient cells as pure DNA (transfection) by, for example, precipitation with calcium phosphate (Graham and vander Eb, 1973, Virology 52:466) strontium phosphate (Brash et al., 1987, Mol. Cell Biol. 7:2013), electroporation (Neumann et al., 1982, EMBO J. 1:841), lipofection (Felgner et al., 1987, Proc. Natl. Acad Sci USA 84:7413), DEAE dextran (McCuthan et al., 1968, J. Natl. Cancer Inst. 41:351), microinjection (Mueller et al., 1978, Cell 15:579), protoplast fusion (Schafner, 1980, Proc. Natl. Acad. Sci. USA 77:2163-7), or pellet guns (Klein et al., 1987, Nature 327:70). Alternatively, the cDNA can be introduced by infection with virus vectors, for example retroviruses (Bernstein et al., 1985, Gen. Engrg. 7:235) such as adenoviruses (Ahmad et al., 1986, J. Virol. 57:267) or Herpes (Spaete et al., 1982, Cell 30:295).

### EXAMPLE 21

### Detection of Src Mutations

One application of the observation that the phosphorylation state of Ser17 affects cell proliferation, is in the area of genetic testing, carrier detection and prenatal diagnosis. For example, subjects having one or more mutations in a Src gene, such as an S17A or S17D mutation or other mutation associated with disease, are detected at the DNA level using a variety of techniques. The presence of one or more nucleotide differences between the subject's sequence and a wild-type Src cDNA sequence, such as differences in the region coding for Ser17, are indicative of a potential Src gene mutation. The phenotypes of subjects in whom the mutation is detected are noted, and the same mutations in other subjects may predict a similar phenotype. If the cells (or the subject from whom the sample is taken) are normal, observed nucleotide differences are regarded as neutral, and the subject is not classified as a carrier or sufferer on the basis of this nucleotide difference. If the altered cDNA reveals an abnormal result, the nucleotide difference is a mutation rather than a neutral difference, the protein is an aberrant (or mutant) Src gene product, and the subject is classified as a sufferer or carrier.

For such a diagnostic procedure, a sample containing DNA or RNA obtained from the subject, is assayed for the presence of a mutant Src gene, using any method known in the art. For example, RT-PCR of RNA isolated from cells, such as lymphocytes, followed by direct DNA sequence determination of the products, can be used. Alternatively, DNA extracted from lymphocytes or other cells is used directly for amplification. Amplification from genomic DNA is appropriate for analysis of the entire Src gene (see Caskey, Science 236:1223-8, 1989; and Landegren et al. Science 242:229-37, 1989).

In another example, rather than sequencing the entire Src gene, DNA diagnostic methods are designed to detect the most common mutations, deletions, or variants, for example those which affect the sequence or phosphorylation state of Src Ser17. The detection of specific DNA mutations can be achieved by hybridization with specific oligonucleotides (Wallace et al., 1986, Cold Spring Harbor Symp. Quant. Biol. 51:257-61), direct DNA sequencing (Church and Gilbert, 1984, Proc. Natl. Acad. Sci. USA. 81:1991-5), restriction enzymes (Flavell et al., 1978, Cell 15:25; Geever et al., 1981, Proc. Natl. Acad. Sci USA 78:5081), discrimination on the basis of electrophoretic mobility in gels with denaturing reagent (Myers and Maniatis, 1986, Cold Spring Harbor Synp. Quant. Biol. 51:275-84), RNase protection (Myers et al., 1985, Science 230:1242), chemical cleavage (Cotton et al., 1985, Proc. Natl. Acad. Sci. USA 85:4397401), and ligase-mediated detection (Landegren et al., 1988, Science 241:1077).

Oligonucleotides specific to normal or mutant sequences can be chemically synthesized, radioactively labeled (such as with ³²P) or non-radioactively labeled (such as with biotin) (Ward and Langer, Proc. Natl. Acad. Sci. USA 78:6633-57, 1981), and hybridized to individual DNA samples immobilized on membranes or other solid supports by dot-blot or transfer from gels after electrophoresis. The presence or absence of specific sequences are visualized by methods such as autoradiography, fluorometric (Landegren et al., 1989, Science 242:229-37) or calorimetric reactions (Gebeyehu et al., 1987, Nucleic Acids Res. 15:4513-34).

Sequence differences between normal, variant, polymorphic, and mutant forms of Src genes can be revealed by direct DNA sequencing (Church and Gilbert, 1988, Proc. Natl. Acad. Sci USA 81:1991-5). Cloned DNA segments can be used as probes to detect specific DNA segments. The sensitivity of this method is greatly enhanced when combined with PCR (Wrichnik et al., 1987, Nucleic Acids Res. 15:529-42; Wong et al., 1987, Nature 330:384-6; Stoflet et al., 1988, Science 239:4914). In this approach, a sequencing primer which lies within the amplified sequence is used with double-stranded PCR product or single-stranded template generated by a modified PCR. The sequence determination is performed by conventional procedures. The absence of hybridization indicates a mutation in a particular region of the gene, or a deleted gene.

Sequence alterations may generate fortuitous or eliminate existing restriction enzyme recognition sites. Changes in restriction sites are revealed by using appropriate enzyme digestion followed by conventional gel-blot hybridization (Southern, 1975, J. Mol. Biol. 98:503). DNA fragments carrying the site (either normal or mutant) are detected by their reduction in size or increase of corresponding restriction fragment numbers. Genomic DNA samples may also be amplified by PCR prior to treatment with the appropriate restriction enzyme; fragments of different sizes are then visualized under UV light in the presence of ethidium bromide after gel electrophoresis.

Genetic testing based on DNA sequence can be achieved by detection of alteration in electrophoretic mobility of DNA fragments in gels with or without denaturing reagent. Small sequence deletions and insertions can be visualized by high-resolution gel electrophoresis. For example, a PCR product with small deletions is clearly distinguishable from a normal sequence on an 8% non-denaturing polyacrylamide gel (WO 91/10734; Nagamine et al, 1989, Am. J. Hum. Genet. 45:337-9). DNA fragments of different sequence compositions may be distinguished on denaturing formamide gradient gels in which the mobilities of different DNA fragments are retarded in the gel at different positions according to their specific partial-melting temperatures (Myers et al., 1985, Science 230:1242). Alternatively, a method of detecting a mutation comprising a single base substitution or other small change can be based on differential primer length in a PCR. For example, an invariant primer can be used in addition to a primer specific for a mutation. The PCR products of the normal and mutant genes are differentially detected in acrylamide gels.

In addition to conventional gel-electrophoresis and blot-hybridization methods, DNA fragments can be visualized by methods where individual DNA samples are not immobilized on membranes. The probe and target sequences may be both in solution, or the probe sequence can be immobilized (Said et al., 1989, Proc. Nat Acad. Sci. USA 86:6230-4). A variety of detection methods, such as autoradiography involving radioisotopes, direct detection of radioactive decay (in the presence or absence of scintillant), spectrophotometry involving caloxigenic reactions and fluorometry involved fluorogenic reactions, can be used to identify specific individual genotypes.

A method suitable for detecting the presence of Src genes disclosed herein is the use of high-density oligonucleotide arrays (also known as “DNA chips”) as described by Hacia et al. (Nat. Genet. 14:441-7, 1996).

The diagnostic assays disclosed herein may be assembled in the form of a diagnostic kit and may include, for example: hybridization with oligonucleotides; PCR amplification of the gene or a part thereof using oligonucleotide primers; RT-PCR amplification of the RNA or a part thereof using oligonucleotide primers; or direct sequencing of a Src gene of the subject's genome using oligonucleotide primers. The efficiency of these molecular genetic methods should permit a rapid classification of patients affected by mutations, deletions or variants of an Src gene.

### EXAMPLE 22

### Peptide Synthesis and Purification

The Src peptides (and variants, fusions, polymorphisms, fragments, and mutants thereof), such as Src peptides phosphorylated at Ser17, can be chemically synthesized by any of a number of manual or automated methods of synthesis known in the art. For example, solid phase peptide synthesis (SPPS) is carried out on a 0.25 millimole (mmole) scale using an Applied Biosystems Model 431A Peptide Synthesizer and using 9-fluorenylmethyloxycarbonyl (Fmoc) amino-terminus protection, coupling with dicyclohexylcarbodiimide/hydroxybenzotriazole or 2-(1H-benzo-triazol-1-yl)-1, 1,3,3-tetramethyluronium hexafluorophosphate/hydroxybenzotriazole (HBTU/HOBT), and using p-hydroxymethylphenoxymethylpolystyrene (HMP) or Sasrin resin for carboxyl-terminus acids or Rink amide resin for carboxyl-terminus amides.

Fmoc-derivatized amino acids are prepared from the appropriate precursor amino acids by tritylation and triphenylmethanol in trifluoroacetic acid, followed by Fmoc derivitization as described by Atherton et al. (Solid Phase Peptide Synthesis, IRL Press: Oxford, 1989).

Sasrin resin-bound peptides are cleaved using a solution of 1% TFA in dichloromethane to yield the protected peptide. Where appropriate, protected peptide precursors are cyclized between the amino- and carboxyl-termini by reaction of the amino-terminal free amine and carboxyl-terminal free acid using diphenylphosphorylazide in nascent peptides wherein the amino acid sidechains are protected.

HMP or Rink amide resin-bound products are routinely cleaved and protected sidechain-containing cyclized peptides deprotected using a solution comprised of trifluoroacetic acid (TFA), optionally also comprising water, thioanisole, and ethanedithiol, in ratios of 100:5:5:2.5, for 0.5-3 hours at RT.

Crude peptides are purified by preparative high-pressure liquid chromatography (HPLC), for example using a Waters Delta-Pak C18 column and gradient elution with 0.1% TFA in water modified with acetonitrile. After column elution, acetonitrile is evaporated from the eluted fractions, which are then lyophilized. The identity of each product so produced and purified may be confirmed by fast atom bombardment mass spectroscopy (FABMS) or electrospray mass spectroscopy (ESMS).

### EXAMPLE 23

### Methods for In Vivo or Ex Vivo Src Expression

The present disclosure provides methods of expressing Src that is phosphorylated at Ser17, or a functional equivalent thereof, such as the S17D mutant disclosed herein which mimics phosphorylation, in a cell or tissue in vivo. Such methods are useful if decreased proliferation of the cell or cells of the tissue is desired, such as for a tumor or cancer. For example, decreased proliferation is desired when subjects have a proliferating tumor that is associated with Src in which phosphorylation at Ser17 is inhibited or otherwise impaired, for example by a mutation at Ser17 of Src.

In addition, the present disclosure provides methods of expressing Src that is not phosphorylated at Ser17, or a functional equivalent thereof, such as the S17A mutant disclosed herein which mimics de-phosphorylation, in a cell or tissue in vivo. Such methods are useful if increased proliferation of the cell or cells of the tissue is desired. For example, increased proliferation is desired in subjects having impaired cell survival, such as damaged myocardium following infarction.

In one example, transfection of the cell or tissue occurs in vitro. In this example, the cell or tissue is removed from a subject and then transfected with an expression vector containing the desired cDNA. The transfected cells will produce functional protein and can be reintroduced into the subject. In another embodiment, a nucleic acid is administered to the subject directly, and transfection occurs in vivo.

The Src sequences and variants disclosed herein can be used in methods of treating a subject with a proliferative disorder such as cancer. Such a method would decrease proliferation, if the Src sequence expressed could be or was phosphorylated at Ser17. However, mutant Src sequences that increase proliferation, such as those that prevent or cannot be phosphorylated at Ser17, can be used to increase the development of muscle cells in persons suffering from, for example heart disease or to increase the development of bone cells in persons suffering from osteoporosis. Src inhibitors have been disclosed for use in methods of promoting angiogenesis and vascular permeability (WO 14575 A1), treating HBV infection and hepatocellular carcinoma (WO 98/57175 A1)

The scientific and medical procedures required for human cell transfection are now routine. The public availability of Src cDNAs, as well as the disclosure herein of the role of the phosphorylation state of Src Ser17, allows the development of human (and other mammals) in vivo gene expression based upon these procedures. Immunotherapy of melanoma patients using genetically engineered tumor-infiltrating lymphocytes (TILs) has been reported by Rosenberg et al. (N. Engl. J. Med. 323: 570-8, 1990). In that study, a retrovirus vector was used to introduce a gene for neomycin resistance into TILs. A similar approach may be used to introduce a Src cDNA into subjects affected by a proliferative disorder.

In some examples, a method of treating subjects which under express functional Src, or in which greater functional Src expression is desired, is disclosed. These methods can be accomplished by introducing a gene coding for Src into a subject, or a gene encoding for the S17D mutant, which mimics phosphorylation. A general strategy for transferring genes into donor cells is disclosed in U.S. Pat. No. 5,529,774, incorporated by reference. Generally, a gene encoding a protein having therapeutically desired effects is cloned into a viral expression vector, and that vector is then introduced into the target organism. The virus infects the cells, and produces the protein sequence in vivo, where it has its desired therapeutic effect Zabner et al. (Cell 75:207-16, 1993).

It may only be necessary to introduce the genetic or protein elements into certain cells or tissues. For example, in the case of cancer, introducing them into only the tumor may be sufficient. However, in some instances, it may be more therapeutically effective and simple to treat all of a subject's cells, or more broadly disseminate the vector, for example by intravascular administration.

The nucleic acid sequence encoding at least one therapeutic agent is under the control of a suitable promoter. Suitable promoters which may be employed include, but are not limited to, the gene's native promoter, retroviral LTR promoter, or adenoviral promoters, such as the adenoviral major late promoter; the CMV promoter; the RSV promoter; inducible promoters, such as the MMTV promoter; the metallothionein promoter; heat shock promoters; the albumin promoter; the histone promoter; the α-actin promoter; TK promoters; B19 parvovirus promoters; and the ApoAI promoter. However the scope of the disclosure is not limited to specific foreign genes or promoters.

The recombinant nucleic acid can be administered to the subject by any method that allows the recombinant nucleic acid to reach the appropriate cells. These methods include injection, infusion, deposition, implantation, or topical administration. Injections can be intradermal or subcutaneous. The recombinant nucleic acid can be delivered as part of a viral vector, such as avipox viruses, recombinant vaccinia virus, replication-deficient adenovirus strains or poliovirus, or as a non-infectious form such as naked DNA or liposome encapsulated DNA, as further described in EXAMPLE 15.

### EXAMPLE 24

### Viral Vectors for in vivo Gene Expression

Viral vectors can be used to express a desired Src sequence in vivo. Methods for using such vectors for in vivo gene expression are well known in the art (for example see U.S. Pat. No. 6,306,652 to Fallaux et al., U.S. Pat. No. 6,204,060 to Mehtali et al., U.S. Pat. No. 6,287,557 to Boursnell et al., and U.S. Pat. No. 6,217,860 to Woo et al., all herein incorporated by reference). Specific examples of such vectors include, but are not limited to: adenoviral vectors; adeno-associated viruses (AAV); retroviral vectors such as MMLV, spleen necrosis virus, RSV, Harvey Sarcoma Virus, avian leukosis virus, HIV, myeloproliferative sarcoma virus, and mammary tumor virus, as well as and vectors derived from these viruses. Other viral transfection systems may also be utilized; including Vaccinia virus (Moss et al., 1987, Annu. Rev. Immunol. 5:305-24), Bovine Papilloma virus (Rasmussen et al., 1987, Methods Enzymol 139:642-54), and herpes viruses, such as Epstein-Barr virus (Margolskee et al., 1988, Mol. Cell. Biol. 8:2837A47). In another example, RNA-DNA hybrid oligonucleotides, as described by Cole-Strauss et al. (Science 273:1386-9, 1996) are used.

Viral particles are administered in an amount effective to produce a therapeutic effect in a subject The exact dosage of viral particles to be administered is dependent upon a variety of factors, including the age, weight, and sex of the subject to be treated, and the nature and extent of the disease or disorder to be treated. The viral particles may be administered as part of a preparation having a titer of viral particles of at least 1×10¹⁰ pfu/ml, and in general not exceeding 2×10¹¹ pfu/ml. The viral particles can be administered in combination with a pharmaceutically acceptable carrier in a volume up to 10 ml. The pharmaceutically acceptable carrier may be, for example, a liquid carrier such as a saline solution, protamine sulfate (Elkins-Sinn, Inc., Cherry Hill, N.J.), or Polybrene (Sigma Chemical) as well as those described in EXAMPLE 25.

### EXAMPLE 25

### Pharmaceutical Compositions and Modes of Administration

The present disclosure also provides pharmaceutical compositions that include a therapeutically effective amount of an agent or compound which specifically enhances or decreases phosphorylation at Ser17 of Src, alone or with a pharmaceutically acceptable carrier. Furthermore, the pharmaceutical compositions or methods of treatment can be administered in combination with other therapeutic treatments, such as other anti-neoplastic agents. Any method of introduction and any pharmaceutical composition that includes a therapeutically effective agent disclosed herein, can be used.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

In an example in which a Src nucleic acid (or fragment, variant, fusion, mutant, or polymorphism thereof), such as the Src S17A or S17D mutant, is used to allow expression of the nucleic acid in a cell, the nucleic acid is delivered intracellularly (e.g., by expression from a nucleic acid vector or by receptor-mediated mechanisms). Administration can be achieved by an appropriate nucleic acid expression vector (see EXAMPLES 23 and 24), direct injection, microparticle bombardment (e.g., a gene gun), transfecting agents, or any other method known in the art.

In an example where the therapeutic molecule is an agent that mimics phosphorylated or unphosphorylated Src Ser17 when administered to a cell, such as a specific-binding agent or mimetic, administration may be achieved by direct injection, microparticle bombardment (e.g., a gene gun), or coating with lipids, cell-surface receptors, transfecting agents. Similar methods can be used to administer a Src protein, or fragments, fusions, mutants, polymorphisms, or variants thereof

### EXAMPLE 26

### Generation and Expression of Fusion Proteins

Src fusion proteins or peptides which include a Src sequence (such as full-length Src, or fragments, mutants, variants, or polymorphisms of Src) can be generated using standard methods known to those skilled in the art (for example see U.S. Pat. No. 6,057,133 to Bauer et al. and U.S. Pat. No. 6,072,041 to Davis et al., both incorporated by reference). In one example, linker regions are used to space the two portions of the protein from each other and to provide flexibility between the two peptides, such as a polypeptide of between 1 and 500 amino acids. Other moieties can also be included, such as a binding region (i.e. avidin or an epitope, such as a polyhistadine tag), which can be useful for purification and processing of the fusion protein. In addition, detectable markers can be attached to the fusion protein.

### EXAMPLE 27

### Transgenic Animals

Animals that express Src, or a variant or mutant thereof such as the S17A or S17D mutant, in their cells can be used to determine in vivo effects of test agents (see Example 18). Examples of transgenic animals include, but are not limited to, mice, rats, primates, and rabbits. For example, transgenic animals which express the S17A Src mutant will have a higher incidence of Src-positive tumors, and/or such tumors grow more rapidly, and/or the animal heals more quickly subsequent to a heart attack, due to increases in cell proliferation. Therefore, such animals are beneficial to researchers who want an animal model that rapidly produces Src-positive tumors. In addition, these animals are useful as an animal model for testing or screening for agents that reverse the effect of the mutation, for example agents that decrease tumor proliferation after administration of the test agent to the animal. The effect of the test agent in treating the in vivo effects of the presence of non-phosphorylated Src Ser17 can be compared to animals not receiving the test agent.

In contrast, trangenic animals which express the S17D mutant will have a lower incidence of Src-positive tumors, due to decreased cell proliferation. In another example, such animals do not have the ability to heal, or heal more slowly, subsequent to a heart attack. These animals are useful as an animal model for testing or screening for agents that reverse the effect of the mutation, that is, agents that increase cell proliferation, such as increase myocardial proliferation and/or and increase in tumor proliferation after administration of the test agent to the animal, are used to investigate in vivo effects of Src phosphorylation at Ser17. The effect of the test agent in treating the in vivo effects of the presence of phosphorylated Src Ser17 can be compared to animals not receiving the test agent.

Methods for generating transgenic mice are described in Gene Targeting, A. L. Joyuner ed, Oxford University Press, 1995 and Watson, J. D. et al., Recombinant DNA 2nd Ed., W. H. Freeman and Co., New York, 1992, Chapter 14 as well as in U.S. Pat. Nos. 5,574,206; 5,723,719; 5,175,383; 5,824,838; 5,811,633; 5,620,881; and 5,767,337, herein incorporated by reference.

Briefly, a vector comprising the desired Src sequence, such as the plasmids and vectors disclosed in EXAMPLES 20 and 24, is generated. The plasmid is linearized, purified, and microinjected into mouse embryos that were then implanted into surrogate mothers. Pups are screened for the presence of the transgene by PCR on tail snippets. Once at least on positive founder male and one positive founder female containing the transgene in their genomes are identified, the founder animals are separately bred into a C57b16 background for several generations until homozygous positive mice are obtained which breed true (generated all positive litters). The phenotypes of the mice, such as their ability to produce Src-positive tumors, can then be determined.

In view of the many possible embodiments to which the principles of our disclosure may be 5 applied, it should be recognized that the illustrated embodiments are only examples of the disclosure and should not be taken as a limitation on the scope of the disclosure. Rather, the scope of the disclosure is defined by the following claims. We therefore claim as our invention all that comes within the scope and spirit of these claims.

1. A specific-binding agent which specifically binds to Src when Src is phosphorylated at serine at position 17 (Ser17), but does not detectably bind to Src when Ser17 is not phosphorylated.

2. The specific-binding agent of claim 1, wherein the specific-binding agent is a drug.

3. The specific-binding agent of claim 1, wherein the specific-binding agent is an antibody.

4. The specific-binding agent of claim 3, wherein the antibody is a polyclonal antibody.

5. The specific-binding agent of claim 3, wherein the antibody is a monoclonal antibody.

6. An immortal cell line that produces the monoclonal antibody of claim 5.

7. The specific-binding agent of claim 3, wherein the antibody is a chimeric antibody.

8. The specific-binding agent of claim 1, wherein the specific binding agent specifically binds to a sequence selected from the group consisting of QRRRSLEPA (SEQ ID NO: 5), SQRRRSLEPAE (SEQ ID NO: 6), ASQRRRSLEPAEN (SEQ ID NO: 7), DASQRRRSLEPAENV (SEQ ID NO: 8), KDASQRRRSLEPAENVH (SEQ ID NO: 9), PKDASQRRRSLEPAENVHG (SEQ ID NO: 10), and KPKDASQRRRMLEPAENVHGA (SEQ ID NO: 11)), wherein an underlined serine is phosphorylated.

9. A method for detecting Src, wherein Src is phosphorylated at Ser17, comprising:
contacting a sample with the specific-binding agent of claim 1 under conditions such that the specific-binding agent will specifically bind to a Src phosphorylated at Ser17 present in the sample to form a specific-binding agent:Src phosphorylated at Ser17 complex; and
detecting the presence or absence of the specific-binding agent:Src phosphorylated at Ser17 complex, wherein the presence of the complex indicates the presence of Src phosphorylated at Ser17 in the sample, which is associated with decreased cell proliferation.

10. The method of claim 9, further comprising quantitating an amount of the specific-binding agent:Src phosphorylated at Ser17 complex.

11. The method of claim 9, wherein the specific-binding agent comprises a label, and wherein detecting the specific-binding agent:Src phosphorylated at Ser17 complex comprises detection of the label.

12. The method of claim 11, wherein the label is a radioisotope, a fluorophore, a bioluminescent compound, an enzyme, or a chemiluminescent compound.

13. The method of claim 9, wherein the sample is a protein sample obtained from a tumor, and absence of the specific-binding agent:Src phosphorylated at Ser17 complex in the sample is associated with increased cell proliferation.

14. The method of claim 13, wherein the tumor is a Src-positive tumor.

15. The method of claim 14, wherein the Src-positive tumor is a tumor of the breast, ovary, colon, brain, pancreas, lung, esophagus, skin, eye, hematopoietic system or gastric tract.

16. The method of claim 14, wherein the Src-positive tumor is a melanoma, adenocarcinoma, retinoblastoma, or neuroblastoma.

17. The method of claim 15, wherein the tumor is a tumor of the breast.

18. The method of claim 9, wherein the specific-binding agent is an antibody.

19. A method of identifying a cell having abnormal proliferation, comprising detecting a mutation in Src at Ser17 which is associated with abnormal cellular proliferation.

20. The method of claim 19, where detection of the mutation in Src at Ser17 comprises amplifying a Src nucleic acid sequence which includes a region encoding Ser17.

21. The method of claim 20, wherein the method is a method for identifying a cell having increased proliferation, comprising detecting a mutation in Src at Ser17 which is associated with increased cellular proliferation.

22. The method of claim 21, wherein the mutation in Src at Ser17 is an S17A mutation, which is associated with decreased phosphorylation at Src Ser17.

23. The method of claim 21, wherein the mutation is further associated with enhanced susceptibility of a subject to a Src-positive tumor.

24. The method of claim 20, wherein the method is a method for identifying a cell having decreased proliferation, comprising detecting a mutation in Src at Ser17 which is associated with mimicking phosphorylation of Src at Ser17.

25. The method of claim 24, wherein the mutation in Src at Ser17 is an S17D mutation, which is associated with decreased cellular proliferation.

26. A method of screening a test agent to identify agents which alter cell proliferation, comprising:
contacting the test agent with a cell in which Src is mutated at Ser17 to alter Ser17 phosphorylation and produce a cell having abnormal proliferation; and
determining whether the test agent alters the abnormal cell proliferation.

27. The method of claim 26, wherein a cell proliferation assay, a Rap1 activation affinity assay, or a Ras activation affinity assay is performed on the cell to determine whether the test agent alters the abnormal cell proliferation.

28. The method of claim 26, wherein the method is a method of screening a test agent to identify agents which decrease cell proliferation and the method comprises determining whether cell proliferation decreases in the presence of the test agent

29. The method of claim 28, wherein the method identifies agents which mimic Src when Src is phosphorylated at Ser17, which decrease cell proliferation.

30. The method of claim 28, wherein the mutation at Ser17 is an S17A mutation, and wherein decreased proliferation indicates that the test agent can compensate for Src mutations at Ser17 which decrease phosphorylation of Ser17.

31. The method of claim 28, wherein the mutation at Ser17 is an ablation of the Src gene, and wherein decreased proliferation indicates that the test agent can compensate for Src mutations which decrease phosphorylation of Ser17.

32. The method of claim 31, wherein the cell is an SYF cell.

33. The method of claim 26, wherein the method is a method of screening the test agent to identify agents which increase cell proliferation and the method comprises determining whether cell proliferation increases in the presence of the test agent.

34. The method of claim 33, wherein the method identifies agents which mimic Src when Src is not phosphorylated at Ser17, which increase cell proliferation.

35. The method of claim 33, wherein the mutation at Ser17 is an S17D mutation, and wherein increased proliferation indicates that the test agent can compensate for Src mutations at Ser17 which increase phosphorylation.

36. A method of screening a test agent to identify agents which alter phosphorylation of Src at Ser17, comprising:
contacting the test agent with a Src peptide comprising Ser17, thereby generating a test sample;
performing a kinase assay on the test sample; and
determining whether there is enhanced Src phosphorylation at Ser17 compared to a test sample which does not include the test agent.

37. The method of claim 36, wherein the method is a method for identifying agents which increase phosphorylation of Src at Ser17, which is associated with decreased cell proliferation.

38. The method of claim 36, wherein the method is a method for identifying agents which decrease phosphorylation of Src at Ser17, which is associated with increased cell proliferation.

39. A method of altering cell proliferation, comprising altering phosphorylation of Src Ser17 in a cell.

40. The method of claim 39, wherein altering phosphorylation of Src Ser17 comprises contacting the cell with an agent which alters phosphorylation of Src Ser17.

41. The method of claim 39, wherein the method is a method of increasing cell proliferation by decreasing phosphorylation of Src Ser17 in the cell.

42. The method of claim 41, wherein decreasing phosphorylation of Src Ser17 comprises expressing a Src S17A mutant in the cell.

43. The method of claim 39, wherein the method is a method of decreasing cell proliferation comprising increasing phosphorylation of Src Ser17 in the cell.

44. The method of claim 43, wherein increasing phosphorylation of Src Ser17 comprises mimicking phosphorylation of Src Ser17 comprising expressing a Src S17D mutant in the cell.

45. The method of claim 40, wherein contacting the cell includes administering an effective amount of the agent to a subject in whom increased or decreased cell proliferation is desired.

46. The method of claim 45, wherein the subject is a mammal.

47. The method of claim 46, wherein the mammal is a human.

48. The method of claim 45, wherein decreased cell proliferation is desired and the agent promotes phosphorylation of Src Ser17.

49. The method of claim 48, wherein the subject has a Src-positive tumor.

50. The method of claim 49, wherein the Src-positive tumor is a tumor of the breast, ovary, colon, brain, eye, skin, pancreas, lung, esophagus, hematopoietic system, or gastric tract.

51. The method of claim 45, wherein increased cell proliferation is desired, and the agent inhibits phosphorylation of Src Ser17.

52. The method of claim 51, wherein the subject has heart disease, Alzheimer's, Parkinson's, or osteoporosis.

53. The method of claim 52, wherein the subject is a laboratory mammal in whom increased proliferation of a Src-positive tumor is desired.

54. The method of claim 41, wherein the cell is a breast cancer cell cultured ex-vivo.

55. The method of claim 44, wherein the method further treats a Src-positive tumor.

56. The method of claim 42, wherein the method further treats heart disease.

57. Use of the specific-binding agent of claim 1 as a diagnostic agent.

58. A transgenic mammal comprising a mutation at Src Ser17 which alters phosphorylation of Src Ser17.

59. The transgenic mammal of claim 58, wherein the mutation inhibits phosphorylation of Src Ser17 and promotes cellular proliferation of cells in which the mutation is present.

60. The transgenic mammal of claim 59, wherein the mutation at Src Ser 17 is a S17A Src mutation.

61. The transgenic mammal of claim 58, wherein the mutation promotes phosphorylation of Src Ser17 or mimics phosphorylation of Src Ser17 and inhibits cellular proliferation of cells in which the mutation is present.

62. The transgenic mammal of claim 61, wherein the mutation at Src Ser17 is a S17D Src mutation.

63. A method of screening for agents which affect cellular proliferation, comprising administration of a test agent to the transgenic mammal of claim 58, and determining whether the agent offsets the altered phosphorylation of Src Ser17.
